(19) United States
(12) Patent Application Publication
(10) Pub. No.: US 2010/0144714 A1
(43) Pub. Date: Jun. 10, 2010

(54) DERIVATIVES OF IMIDAZO PYRIMIDO AND DIAZEPINE PYRIMIDINE-DIONE, AND USE THEREOF AS A DRUG

(75) Inventors: Lydie Poitout, Antony (FR); Valérie Brault, Saint-Arnoult-En-Yvelines (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SOCIETE DE CONSEILS DE RECHERCHES ET D'APPLICATIONS SCIENTIFIQUES (S.C.R.A.S.), Paris (FR)

(21) Appl. No.: 12/519,647

(22) PCT Filed: Dec. 17, 2007
(86) PCT No.: PCT/FR2007/002090

§ 371 Date: Feb. 16, 2010

(30) Foreign Application Priority Data
Dec. 18, 2006 (FR) 0611001

(51) Int. Cl. A61K 31/496; C07D 487/04; A61K 31/551; A61P 25/16; A61P 25/14

(52) U.S. Cl. 514/220; 544/250; 544/230; 540/497; 514/252.16; 514/252.15

(57) ABSTRACT

The present invention relates to new derivatives of imidazo, pyrimido and diazepine-pyrimidine-dione of the general formula (I) in which R₁, R₂, L₁, L₂, Y, Z and A are various and varying groups. These products exhibit a good affinity for certain sub-types of cannabinoid receptors, in particular the CB2 receptors. They are particularly useful for treating pathological conditions and diseases in which one or more cannabinoid receptors are involved. The invention also relates to pharmaceutical compositions containing said products, and to the use thereof for preparing a drug.

The subject of the present application is novel derivatives of imidazo, pyrimido and diazepine pyrimidine-dione. These products have a good affinity for certain sub-types of cannabinoid receptors, in particular the CB2 receptors. They are particularly useful for treating the pathological states and diseases in which one or more cannabinoid receptors are involved. The invention also relates to pharmaceutical compositions containing said products and their use for the preparation of a medicament.

The cannabinoids are psychoactive components present in Indian cannabis (Cannabis sativa) containing approximately 6 different molecules, the most represented of which is delta-9-tetrahydrocannabinol. Knowledge of the therapeutic activity of cannabis goes back to the ancient Chinese dynasties in which, 5000 years ago, cannabis was used for the treatment of asthma, migraines and gynaecological disorders. It was in 1850 that cannabis extracts were recognized and included in the American pharmacopeia.

The cannabinoids are known for having different effects on numerous functions and organs, the most important being on the central nervous system and on the cardiovascular system. These effects include alterations to the memory, euphoria and sedation. The cannabinoids also increase the pulse and modify the systemic arterial pressure. Peripheral effects linked to bronchial constriction, immunomodulation and inflammation have also been observed. More recently, it has been shown that the cannabinoids suppress the cellular and humoral immune responses and possess anti-inflammatory properties. Despite all of these properties, the therapeutic use of the cannabinoids is controversial because of their psychoactive effects (cause of dependency) but also for their multiple side effects which have not yet been completely characterized. Although numerous works have been carried out in this field since the 1940s, little information existed on the characterization of cannabinoid receptors, the existence of endogenous ligands and until recently on selective products of a particular receptor sub-type.

Two cannabinoid receptors have been identified and cloned, CB1 and CB2. CB1 is expressed predominantly in the central nervous system whereas CB2 is expressed in the peripheral tissues, mainly in the immune system. These two receptors are members of the family of the receptors coupled to the G proteins and their inhibition is linked to the activity of adenylate cyclase.

On the basis of all this information, a need exists for compounds capable of selectively modulating the cannabinoid receptors and therefore the pathologies associated with such receptors. Thus, CB2 modulators offer a single pharmacotherapeutical approach against immune disorders, inflammation, osteoporosis, renal ischaemia and other pathological states. There is a considerable interest in developing cannabinoid analogues having a strong affinity for the CB2 receptor. Cannabinoid analogues which specifically modulate the CB2 receptor, directly or indirectly, can produce clinically useful effects without affecting the central nervous system, thus providing a rational therapeutic approach for a wide variety of pathological states.

The novel compounds of this invention modulate the activity of CB2 and are therefore useful for the treatment and prevention of the pathological states and diseases associated with the activity of the cannabinoid receptors such as, but not limited to, cell proliferation disorders such as cancer, immune disorders, inflammation, pain, osteoporosis, atherosclerosis, epilepsy, nausea associated with chemotherapy treatments, fibrosis, gastro-intestinal disorders, neurodegenerative diseases including multiple sclerosis and dyskinesia, Parkinson's disease, Huntington's chorea, Alzheimer's disease and also for preventing or curing diseases associated with motor function such as Tourette's syndrome, and providing neuroprotection.

A subject of the invention is therefore compounds of general formula (I)

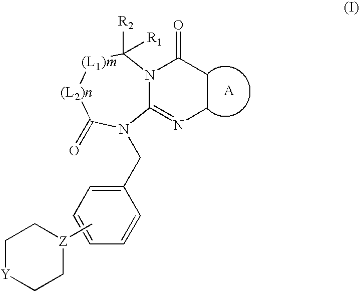

in racemic or enantiomeric form or any combinations of these forms and in which
L₁ represents

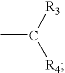

L₂ represents

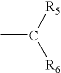

R₂, R₃, R₄, R₅ and R₆ represent, independently, a hydrogen atom or a hydroxy, halo, (C₁-C₈)alkyl, (C₁-C₈)haloalkyl, (C₂-C₈)alkenyl, (C₂-C₈)alkynyl, (C₃-C₈)cycloalkyl, (C₃-C₇)heterocycloalkyl, (C₁-C₆)alkoxy-(C₁-C₈)alkyl, (C₁-C₆)alkylthio-(C₁-C₈)alkyl, aryl, heteroaryl, aralkyloxy, aralkylthio radical or a —(CH₂)ₚ—R₇, radical, all these radicals being optionally substituted by one or more identical or different substituents chosen from: halo, nitro, hydroxy, oxo, amino, carboxamide, (C₁-C₆)alkylcarbonyl, (C₁-C₈)alkyl, (C₁-C₈)haloalkyl, (C₁-C₈)alkoxy, (C₁-C₈)haloalkoxy, aryl or aryloxy;
or R₁ and R₂, R₃ and R₄, or R₅ and R₆, together with the carbon atom to which they are attached, form a (C₃-C₈)cycloalkyl, (C₃-C₇)heterocycloalkyl, indanyl, tetrahydronaphthyl or decahydronaphthyl radical, these radicals being optionally substituted by one or more identical or different substituents chosen from: halo, hydroxy, (C₁-C₈)alkyl, (C₁-C₈)haloalkyl, (C₁-C₆)alkoxy;
or finally the R₃ and R₄ radicals, together with the adjacent R₁ and R₂ radicals on the one hand or R₅ and R₆ radicals on the other hand, form a cycloalkenyl, heterocycloalkenyl, aryl or heteroaryl radical, these radicals being optionally substituted by one or more identical or different substituents chosen from halo, nitro, hydroxy, amino, carboxamide, (C₁-C₆)alkylcarbonyl, (C₁-C₈)alkyl, (C₁-C₈)haloalkyl, (C₁-C₈)alkoxy, (C₁-C₈)haloalkoxy;
p represents 1, 2, 3 or 4;
R₇ represents a —C(O)—O—(C₁-C₈)alkyl, —C(O)—NR_{N}R′_{N}, (C₃-C₈)cycloalkyl, (C₃-C₇)heterocycloalkyl, aryl or heteroaryl radical, all these radicals being optionally substituted by one or more identical or different substituents chosen from: halo, nitro, hydroxy, amino, (C₁-C₈)alkyl, (C₁-C₈)haloalkyl, (C₁-C₈)alkoxy, (C₁-C₈)haloalkoxy or aralkyloxy;
R_{N} and R′_{N} represent independently a hydrogen atom or a (C₁-C₈)alkyl, (C₁-C₈)haloalkyl, (C₂-C₈)alkenyl, (C₂-C₈)alkynyl, (C₃-C₈)cycloalkyl, (C₃-C₇)heterocycloalkyl, aryl or heteroaryl radical;
or R_{N} and R′_{N} together form a (C₃-C₇)heterocycloalkyl radical;
Z represents a nitrogen atom or a —CH— radical;
Y represents an oxygen atom, a —CHR₈ or —NR₈ radical;
R₈ represents a hydrogen atom, a (C₁-C₆)alkyl or (C₁-C₆)haloalkyl radical;
A represents an aromatic or non-aromatic, unsaturated, condensed, mono- or bi-cyclic ring, optionally containing one or more identical or different heteroatoms chosen from O, S and N, and optionally substituted by one or more identical or different radicals, chosen from: halo, nitro, cyano, oxy, —X_{A}—Y_{A}, and aryl optionally substituted by one or more substituents chosen from: halo, (C₁-C₆)alkyl and (C₁-C₆)haloalkyl;
X_{A}, represents a covalent bond, —O—, —S—, —C(O)—, —NR″_{N}—C(O)—, —C(O)—NR″_{N}—, —C(O)—O—, —SO₂— or —SO₂NH—;
Y_{A} represents the hydrogen atom or a (C₁-C₆)alkyl or (C₁-C₆)haloalkyl radical;
R″_{N} represents the hydrogen atom or a (C₁-C₆)alkyl radical;
   m represents 0 or 1; n represents 0 or 1;
   or a pharmaceutically acceptable salt thereof.

In the definitions indicated above, the expression halo represents the fluoro, chloro, bromo or iodo, preferably chloro, fluoro or bromo radical. The expression (C₁-C₈)alkyl represents an alkyl radical having 1 to 8 carbon atoms, linear or branched, such as the methyl, ethyl, propyl, isopropyl, butyl, isobutyl, sec-butyl and tert-butyl, pentyl or amyl, isopentyl, neopentyl, hexyl or isohexyl, heptyl or octyl radicals. The expression (C₁-C₆)alkyl represents a linear or branched alkyl radical having 1 to 6 carbon atoms as defined above.

By (C₂-C₈)alkenyl is meant a linear or branched hydrocarbon radical containing 2 to 8 carbon atoms and having at least one unsaturation (double bond), such as for example vinyl, allyl, propenyl, butenyl, pentenyl, hexenyl or heptenyl. By (C₂-C₈)alkynyl is meant a linear or branched alkyl radical containing 2 to 8 carbon atoms and having at least one double unsaturation (triple bond) such as for example an ethynyl, propargyl, butynyl or pentynyl radical.

By haloalkyl is meant an alkyl radical at least one (and optionally all) of the hydrogen atoms of which is replaced by a halogen atom (halo); in the case of several halo radicals, the latter can be identical such as for example trifluoromethyl or different. The term alkyl-carbonyl (or alkyl-C(O)—) designates the radicals in which the alkyl radical is as defined above, for example methylcarbonyl, ethylcarbonyl, butylcarbonyl.

The term alkoxy designates the radicals in which the alkyl radical is as defined above such as for example the methoxy, ethoxy, propyloxy or isopropyloxy radicals and also secondary or tertiary linear butoxy, pentyloxy. By haloalkoxy is meant an alkoxy radical at least one (and optionally all) of the hydrogen atoms of which is replaced by a halogen atom (halo); in the case of several halo radicals, the latter can be identical such as for example trifluoromethoxy or different. By alkoxy-alkyl is meant a radical in which the alkoxy and alkyl radicals are as defined above such as for example methoxy-ethyl, methoxy-methyl, ethoxy-ethyl. The term alkylthio designates the radicals in which the alkyl radical is as defined above such as for example methylthio, ethylthio. By alkylthio-alkyl is meant a radical in which the alkylthio and alkyl radicals are as defined above such as for example methylthio-ethyl, methylthio-methyl, ethylthio-ethyl.

The term (C₃-C₃)cycloalkyl designates a saturated carbon-containing monocyclic system comprising 3 to 8 carbon atoms, namely the cyclopropyl, cyclobutyl, cyclopentyl, cyclohexyl, cycloheptyl or cyclooctyl rings. The expression (C₃-C₇)heterocycloalkyl designates a condensed saturated monocyclic or bicyclic system containing 3 to 7 carbon atoms and at least one heteroatom. This radical can contain several identical or different heteroatoms. Preferably, the heteroatoms are chosen from oxygen, sulphur or nitrogen. As examples of heterocycloalkyl, the following rings can be mentioned: azetidine, pyrrolidine, imidazolidine, pyrrazolidine, isothiazolidine, thiazolidine, isoxazolidine, oxazolidine, piperidine, piperazine, morpholine, azepane (azacycloheptane), tetrahydrofuran (tetrahydrofuryl radical), tetrahydropyran, dioxane, dioxolane or tetrahydrothiophene (tetrahydrothienyl radical).

The term (C₃-C_{g})cycloalkenyl designates an unsaturated carbon-containing monocyclic system comprising 3 to 8 carbon atoms and a double bond, such as for example cyclopentenyl or cyclohexenyl. The term (C₃-C₇)heterocycloalkenyl designates an unsaturated carbon-containing monocyclic system comprising 3 to 7 carbon atoms, a carbon-carbon double bond and one or more identical or different heteroatoms chosen from sulphur, nitrogen or oxygen such as for example tetrahydropyridine, tetrahydropyrimidine, dihydrofuran, dihydropyrrole, dihydrofuran or dihydrothiophene.

The expression aryl represents an aromatic radical constituted by a condensed ring or rings, such as for example the phenyl, naphthyl, fluorenyl or anthryl radical. The expression heteroaryl designates an aromatic radical, constituted by a condensed ring or rings, with at least one ring containing one or more identical or different heteroatoms chosen from sulphur, nitrogen or oxygen. As examples of heteroaryl radicals, the following radicals can be mentioned: pyrrolyl, imidazolyl, pyrazolyl, isothiazolyl, thiazolyl, isoxazolyl, oxazolyl, oxadiazolyl, triazolyl, thiadiazolyl, pyridyl, pyrazinyl, pyrimidyl, pyridazinyl, quinolyl, isoquinolyl, quinoxalinyl, indolyl, benzotriazolyl, benzothiazolyl, benzoxadiazoyl, carbazolyl, phenoxazinyl, thieno-pyridinyl (thieno[2,3-b]pyridine, thieno[3,2-b]pyridine, thieno[2,3-c]pyridine, thieno[3,2-c]pyridine, thieno[3,4-b]pyridine, thieno[3,4-c]pyridine), thieno-pyrazinyl (thieno[2,3-b]pyrazine, thieno[3,4-b]pyrazine), thienyl, benzothienyl, furyl, benzofuryl, dihydrobenzofuryl, thioxanthenyl, pyranyl, benzopyranyl, dibenzopyrazinyl, acridinyl.

The term aryloxy preferably designates the radicals in which the aryl radical is as defined above; as examples of aryloxy, phenoxy or naphthyloxy can be mentioned. The term aralkoxy (aralkyl-oxy or aryl-alkoxy) preferably designates the radicals in which the aryl and alkoxy radicals are as defined above, such as for example benzyloxy or phenethyloxy. The term aralkylthio (or aryl-alkylthio) preferably designates the radicals in which the aryl and alkylthio radicals are as defined above, such as for example benzylthio.

The expression aromatic, unsaturated, condensed, mono- or bi-cyclic radical can be illustrated either by the aryl radical as defined above when said aromatic radical contains no heteroatom, or by the heteroaryl radical as defined above when said aromatic radical contains at least one heteroatom.

The expression non-aromatic, unsaturated, condensed, mono- or bi-cyclic radical and containing no heteroatom, can be illustrated by cyclopentenyl or cyclohexenyl.

The expression non-aromatic unsaturated, condensed, mono- or bi-cyclic radical and containing at least one heteroatom can be illustrated by the heteroaryl radicals as defined above and in which at least one double bond is hydrogenated. The radicals associated with the following rings can thus be mentioned as examples: dihydroindolyl, dihydrothiophene (2,5-dihydrothiophene, 2,3-dihydrothiophene), tetrahydropyridine (2,3,4,5-tetrahydropyridine, 1,2,3,6-tetrahydropyridine, 1,2,3,4-tetrahydropyridine), tetrahydro-thieno-pyridine (4,5,6,7-tetrahydro-thieno[3,2-b]pyridine, 4,5,6,7-tetrahydro-thieno[2,3-c]pyridine, 4,5,6,7-tetrahydro-thieno[3,2-c]pyridine), tetrahydropyrimidine (2,3,4,5-tetrahydropyrimidine, 1,2,3,4-tetrahydropyrimidine, 1,4,5,6-tetrahydropyrimidine), tetrahydrobenzothiophene (4,5,6,7-tetrahydro-1-benzothiophene), dihydrocyclopentathiophene (5,6-dihydro-4H-cyclopenta[b]thiophene, benzodioxole, dihydro-benzodioxine.

A more particular subject of the present invention is compounds of formula I as defined above, characterized in that A represents a ring chosen from: phenyl, naphthyl, thienyl, furyl, pyrrolyl, benzothienyl, benzofuryl, thieno-pyridinyl, indolyl and tetrahydrobenzo-thienyl; or a pharmaceutically acceptable salt thereof.

A more particular subject of the present invention is compounds of formula I as defined above, characterized in that A is optionally substituted by one or more identical or different radicals chosen from: halo, nitro, —X_{A}—Y_{A}, and phenyl;
X_{A} represents a covalent bond, —O— or —C(O);
Y_{A} represents the hydrogen atom or a (C₁-C₆)alkyl or (C₁-C₆)haloalkyl radical; or a pharmaceutically acceptable salt thereof.

Very preferably, A represents a phenyl radical optionally substituted by one or more identical or different (C₁-C₆)alkyl or (C₁-C₆)alkoxy radicals; or a pharmaceutically acceptable salt thereof.

A more particular subject of the present invention is also compounds of formula I as defined above, characterized in that Z represents a nitrogen atom; or a pharmaceutically acceptable salt thereof.

A more particular subject of the present invention is compounds of formula I as defined above characterized in that Y represents the —NR₈ radical and R₈ represents a (C₁-C₆)alkyl radical; or a pharmaceutically acceptable salt thereof.

A more particular subject of the present invention is also compounds of formula I as defined above, characterized in that they correspond to the following formula:

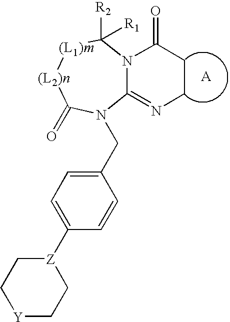

or a pharmaceutically acceptable salt thereof.

A more particular subject of the present invention is also compounds of formula I as defined above, characterized in that n represents 0; or a pharmaceutically acceptable salt thereof.

A more particular subject of the present invention is also compounds of formula I as defined above, characterized in that m represents 0; or a pharmaceutically acceptable salt thereof.

A more particular subject of the present invention is also compounds of formula I as defined above, characterized in that
m and n represent 0;
R₁ represents the hydrogen atom;
R₂ represents a hydrogen atom or a (C₁-C₈)alkyl, (C₃-C₈)cycloalkyl, (C₃-C₇)heterocycloalkyl, aryl radical optionally substituted by an aryl, heteroaryl radical, or a —(CH₂)ₚ—R₇ radical;
p represents 1 or 2;
R₇ represents a (C₃-C₈)cycloalkyl or aryl radical;
or R₁ and R₂, together with the carbon atom to which they are attached, form a (C₃-C₈)cycloalkyl or indanyl radical;
   and preferably
R₁ represents the hydrogen atom;
R₂ represents a (C₁-C₈)alkyl, (C₃-C₈)cycloalkyl, tetrahydropyranyl, naphthyl, phenyl radical optionally substituted by a phenyl, thienyl radical, or a —(CH₂)ₚ—R₇ radical;
   p represents 1 or 2;
   R₇ represents a cyclohexyl or phenyl radical;
or R₁ and R₂, together with the carbon atom to which they are attached, form a (C₃-C₈)cycloalkyl or indanyl radical; or a pharmaceutically acceptable salt thereof.

A more particular subject of the present invention is also compounds of formula I as defined above, characterized in that m represents 1; or a pharmaceutically acceptable salt thereof.

A more particular subject of the present invention is also compounds of formula I as defined above, characterized in that
n represents 0 and m represents 1;
   either R₁, R₂, R₃ and R₄ represent, independently, a hydrogen atom;
   or R₃ and R₄ represent, independently, a hydrogen atom; and
      R₁ represents the hydrogen atom; R₂ represents a (C₁-C₈)alkyl, (C₃-C₈)cycloalkyl, tetrahydropyranyl, phenyl radical, or a —(CH₂)ₚ—R₇ radical; p represents 1 or 2 and R₇ represents a cyclohexyl or phenyl radical; or
      R₁ and R₂ represent, independently, a (C₁-C₈)alkyl radical; or
      R₁ and R₂, together with the carbon atom to which they are attached, form a (C₃-C₈)cycloalkyl.
   or R₁ and R₂ represent, independently, a hydrogen atom;
      R₃ represents the hydrogen atom; R₄ represents a (C₁-C₈)alkyl, (C₃-C₈)cycloalkyl, tetrahydropyranyl, phenyl radical, or a —(CH₂)ₚ—R₇ radical; p represents 1 or 2 and R₇ represents a cyclohexyl radical; or
      R₃ and R₄ represent, independently, a (C₁-C₈)alkyl radical; or
      R₃ and R₄, together with the carbon atom to which they are attached, form a (C₃-C₈)cycloalkyl.
   or the R₃ and R₄ radicals, together with the adjacent R₁ and R₂ radicals, form a phenyl radical; or a pharmaceutically acceptable salt thereof.

A more particular subject of the present invention is also compounds of formula I as defined above characterized in that n and m represent 1; or a pharmaceutically acceptable salt thereof.

A more particular subject of the present invention is also compounds of formula I as defined above, characterized in that
n and m represent 1;
R₁, R₂, R₅ and R₆ represent, independently, a hydrogen atom or a (C₁-C₈)alkyl radical;
R₃ and R₄ represent, independently, a hydrogen atom or, together with the carbon atom to which they are attached, form a (C₃-C₈)cycloalkyl; or a pharmaceutically acceptable salt thereof.

In the present application, the symbol ->* corresponds to the attachment point of the radical. When the attachment site is not specified on the radical, this means that the attachment is carried out on one of the sites of this radical available for such attachment.

According to the definitions of the variable A, Y, Z, R₁, R₂, R₃, R₄, R₅, R₆ and R₇ groups, the compounds according to the invention can be prepared according the procedures A to E described below:

Preparation according to reaction diagram A (case where m and n represent 0):

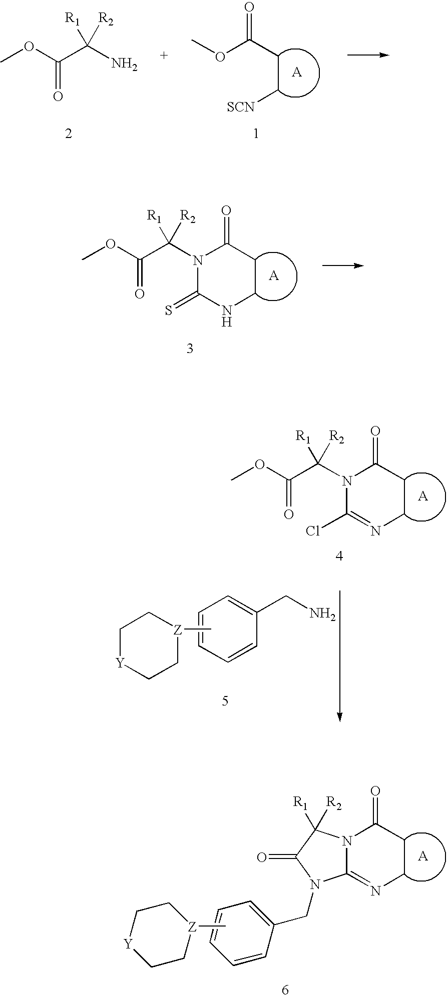

As described in Diagram A, the α-amino-ester (2) can react with an ortho-ester isothiocyanate (1) in a polar solvent such as methanol in the presence or absence of a tertiary amine such as triethylamine at a temperature of 18 to 50° C., for 3 to 24 hours in order to produce the thioxo-pyrimidinone derivative (3). The derivative (3) can be chlorinated by reaction with phosphorus oxychloride in the presence or absence of phosphorus pentachloride, at ambient temperature or heated by microwaves to a temperature of 100 to 150° C. The chlorinated derivative (4) can react with the amine (5) in an aprotic solvent such as tetrahydrofuran in the presence or absence of an inorganic base or tertiary amine such as triethylamine, at ambient temperature or heated by microwaves to a temperature of 80 to 120° C. in order to produce the imidazo-pyrimidine-dione derivative (6).

### EXAMPLE A1

### 1′-[4-(4-methylpiperazin-1-yl)benzyl]-2′H-spiro[cyclopentane-1,3′-imidazo[2,1-b]quinazoline]-2′,5′(1′H)-dione hydrochloride

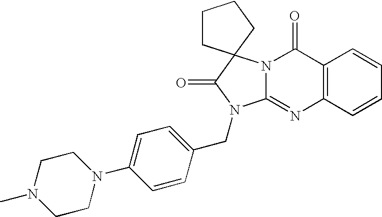

### Stage 1: methyl 1-(4-oxo-2-thioxo-1,4-dihydroquinazolin-3(2H)-yl) cyclopentane carboxylate

A mixture of methyl 1-aminocyclopentanecarboxylate hydrochloride (1 g), methyl 2-isothiocyanatobenzoate (1.18 g) and triethylamine (15 mL) in methanol (60 ml) is stirred at ambient temperature for 20 hours then concentrated under reduced pressure at 40° C. Dichloromethane and water are added to the solid obtained. After decantation and extractions, the combined organic phases are washed with salt water, dried over Na₂SO₄ then concentrated under reduced pressure at 40° C. Purification by flash chromatography on silica gel (eluent: heptane/ethyl acetate 7:3 to 6:4) produces the expected compound in the form of powder (1.08 g, 64% yield).

MS/LC: calculated MM=304.4; m/z=305.2 (MH+)

NMR (¹H, 400 MHz, DMSO-d₆): δ 1.76-1.95 (m, 6H), 2.07 (m, 2H), 3.73 (s, 3H), 7.42 (AB, 1H), 7.60 (m, 1H), 7.74 (m, 1H), 7.99 (AB, 1H), 10.8 (s, 1H).

### Stage 2: methyl 1-(2-chloro-4-oxoquinazolin-3(4H)-yl)cyclopentanecarboxylate

Phosphorus pentachloride (375 mg) is added to a suspension of methyl 1-(4-oxo-2-thioxo-1,4-dihydroquinazolin-3(2H)-yl) cyclopentane carboxylate (500 mg) in phosphorus oxychloride (2 mL). The mixture is stirred for 18 hours at ambient temperature then concentrated under reduced pressure at 40° C. Dichloromethane and water are added to the residue. After decantation and extractions, the combined organic phases are washed with salt water, dried over Na₂SO₄ then concentrated under reduced pressure at 40° C. to produce the expected product in the form of a yellow oil, used in the following stage without subsequent purification.

### Stage 3: 1′-[4-(4-methylpiperazin-1-yl)benzyl]-2′H-spiro[cyclopentane-1,3′-imidazo[2,1-b]quinazoline]-2′,5′(1′H)-dione hydrochloride

[4-(4-methylpiperazin-1-yl)benzyl]amine (950 mg) is added to methyl 1-(2-chloro-4-oxoquinazolin-3(4H)-yl)cyclopentane carboxylate (520 mg) in anhydrous THF (2 mL) placed in a “Biotage®” reaction tube. The reaction tube is sealed with a cap, stirred at ambient temperature for 1 hour then placed in a “Biotage®” micro-wave and heated under magnetic stirring at 100° C. for 1 hour. The mixture is concentrated under reduced pressure at 40° C. Purification by flash chromatography on silica gel (eluent: dichloromethane/methanol 95:5) produces the expected compound in the form of the free base. The corresponding hydrochloride salt is formed by adding a 1N HCl solution in ethyl ether to the solution of the free base in ethyl acetate. The precipitate obtained is filtered and dried in order to produce the expected hydrochloride compound (235 mg, 27% yield from Stage 2).

MS/LC: calculated MM=443.5; m/z=444.3 (MH+)

NMR (¹H, 400 MHz, CDCl₃): δ2.07 (s, 2H), 2.19 (s, 6H), 2.90 (s, 3H), 3.61 (m, 6H), 4.06 (m, 2H), 5.55 (s, 2H), 7.27 (m, 2H), 7.46 (m, 1H), 7.77 (m, 3H), 8.24 (AB, 1H), 8.68 (AB, 1H), 13.35 (s, 1H).

### EXAMPLE A2

### 3-isobutyl-1-[4-(4-methylpiperazin-1-yl)benzyl]imidazo[2,1-b]quinazoline-2,5(1H,3H)-dione hydrochloride

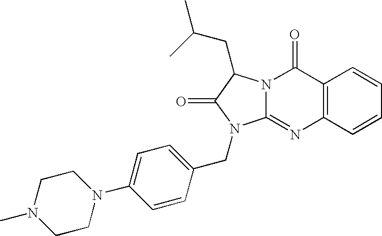

### Stage 1: methyl 4-methyl-2-(4-oxo-2-thioxo-1,4-dihydroquinazolin-3(2H)-yl) pentanoate

A mixture of methyl leucinate hydrochloride (2 g), methyl 2-isothiocyanatobenzoate (2 g) and triethylamine (20 mL) in methanol (60 ml) is stirred at ambient temperature for 24 hours then concentrated under reduced pressure at 40° C. Dichloromethane and water are added to the solid obtained. After decantation and extractions, the combined organic phases are washed with salt water, dried over Na₂SO₄ then concentrated under reduced pressure at 40° C. Purification by flash chromatography on silica gel (eluent: 100% heptane to heptane/ethyl acetate 4:6) produces the expected compound in the form of white foam (3.4 g, 92% yield).

MS/LC: calculated MM=306.4; m/z=307.2 (MH+)

NMR (¹H, 400 MHz, DMSO-d₆): δ0.85 (d, 3H), 0.95 (d, 3H), 1.67 (s, 1H), 1.83 (s, 1H), 2.18 (m, 1H), 3.59 (s, 3H), 6.54 (s, 1H), 7.35 (t, 1H), 7.41 (d, 1H), 7.76 (t, 1H), 7.93 (d, 1H).

### Stage 2: methyl 2-(2-chloro-4-oxoquinazolin-3(4H)-yl)-4-methylpentanoate

Phosphorus pentachloride (2.5 g) is added to a solution of methyl 4-methyl-2-(4-oxo-2-thioxo-1,4-dihydroquinazolin-3(2H)-yl) pentanoate (3.4 g) in phosphorus oxychloride (20 mL). The mixture is stirred at ambient temperature for 20 hours then concentrated under reduced pressure at 40° C. Dichloromethane and water are added to the residue. After decantation and extractions, the combined organic phases are washed with salt water, dried over Na₂SO₄ then concentrated under reduced pressure at 40° C. in order to yield the expected product in the form of a yellow oil, used in the following stage without subsequent purification.

NMR (¹H, 400 MHz, DMSO-d₆): δ0.86 (d, 3H), 0.95 (d, 3H), 1.57 (m, 1H), 1.99 (m, 1H), 2.20 (m, 1H), 3.65 (s, 3H), 7.62 (t, 1H), 7.67 (d, 1H), 7.91 (t, 1H), 8.12 (d, 1H).

### Stage 3: 3-isobutyl-1-[4-(4-methylpiperazin-1-yl)benzyl]imidazo[2,1-b]quinazoline-2,5(1H,3H)-dione hydrochloride

[4-(4-methylpiperazin-1-yl)benzyl]amine (194 mg) is added to methyl 2-(2-chloro-4-oxoquinazolin-3(4H)-yl)-4-methylpentanoate (150 mg) in anhydrous THF (2 mL) placed in a “Biotage®” reaction tube. The tube is sealed with a cap, stirred at ambient temperature for 1 hour then placed in a “Biotage®” micro-wave and heated under magnetic stirring at 150° C. for 1 hour. The mixture is concentrated under reduced pressure at 40° C. Purification by flash chromatography on silica gel (eluent: dichloromethane/methanol 95:5) produces the expected compound in the form of the free base. The corresponding hydrochloride salt is formed by adding a 1N HCl solution in ethyl ether to the solution of the free base in ethyl acetate. The precipitate obtained is filtered and dried in order to produce the expected hydrochloride compound (145 mg, 60% yield from Stage 2).

MS/LC: calculated MM=445.6; m/z=446.3 (MH+)

NMR (¹H, 400 MHz, DMSO-d₆): δ0.81 (m, 6H), 1.80 (m, 1H), 2.01 (m, 2H), 2.77 (s, 3H), 3.06 (m, 4H), 3.40 (m, 2H), 3.77 (m, 2H), 4.79 (AB, 1H), 4.81 (AB, 1H), 4.93 (t, 1H), 6.96 (AB, 1H), 7.32 (AB, 1H), 7.42 (t, 1H), 7.57 (AB, 1H), 7.76 (t, 1H), 8.08 (AB, 1H), 10.96 (s, 1H).

In a fashion analogous to the procedure described for 1′-[4-(4-methylpiperazin-1-yl)benzyl]-2′H-spiro[cyclopentane-1,3′-imidazo[2,1-b]quinazoline]-2′,5′(1′H)-dione hydrochloride and 3-isobutyl-1-[4-(4-methyl piperazin-1-yl)benzyl]imidazo[2,1-b]quinazoline-2,5(1H,3H)-dione hydrochloride, the following compounds were prepared:

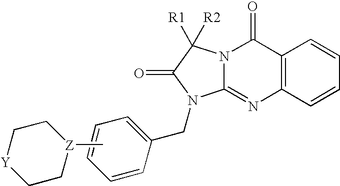

in which R₂ represents the hydrogen atom and R₁ represents one of the radicals below:

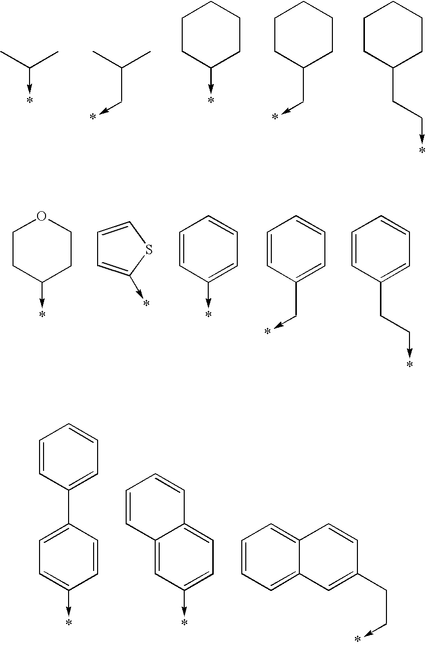

or in which R₁ and R₂ represent the same radical chosen from methyl, propyl and butyl;
or in which R₁ and R₂ together form one of the radicals below:

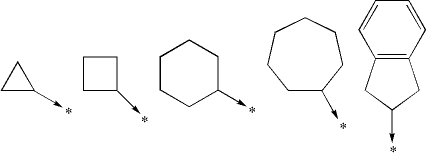

and in which

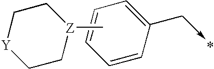

represents one of the radicals below:

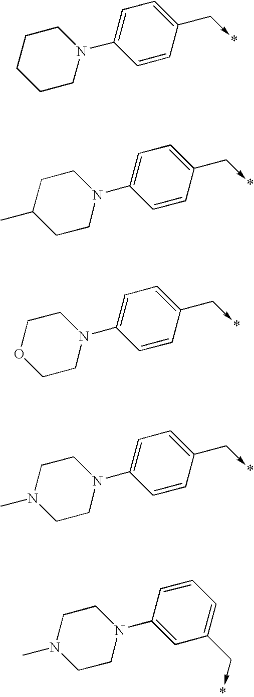

Preparation according to reaction diagram B (case where m and n represent 0):

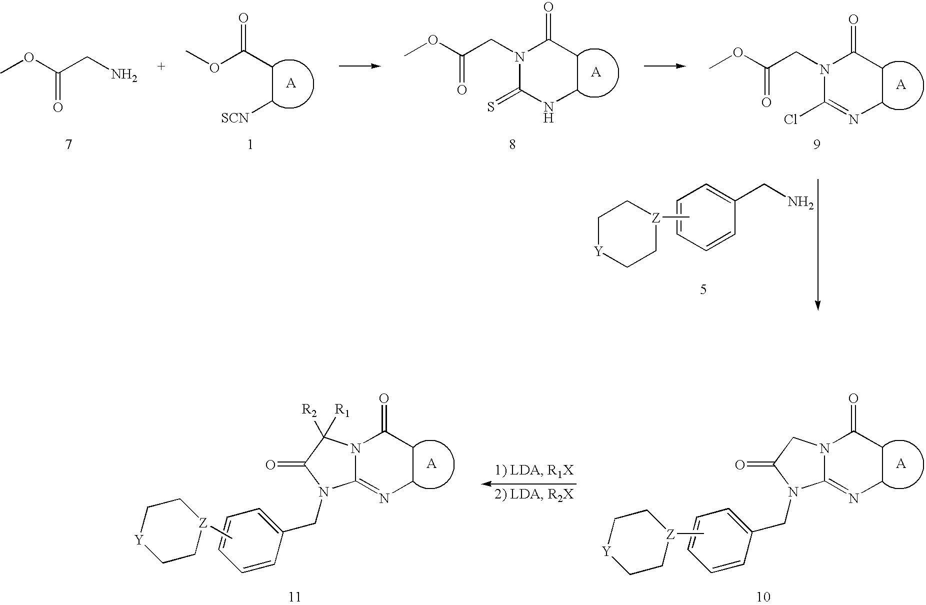

As described in Diagram B, methylglycinate (7) can react with an ortho-ester isothiocyanate (1) in a polar solvent such as methanol in the presence or absence of a tertiary amine such as triethylamine at a temperature of 18 to 50° C., for 3 to 24 hours in order to produce the thioxo-pyrimidinone derivative (8). The derivative (8) can be chlorinated by reaction with phosphorus oxychloride in the presence or absence of phosphorus pentachloride, at ambient temperature or heated by microwaves to a temperature of 100 to 150° C. The chlorinated derivative (9) can react with the amine (5) in an aprotic solvent such as tetrahydrofuran in the presence or absence of an inorganic base or tertiary amine such as triethylamine, at ambient temperature or heated by microwaves to a temperature of 80 to 120° C. in order to produce the imidazo-pyrimidine-dione derivative (10). The derivative (10) can be treated with a strong base such as lithium diisopropylamine followed by alkylation with a halogenated derivative. The reaction can be repeated with a second halogenated derivative in order to produce the derivative (11).

Preparation according to reaction diagram C (case where m represents 1 and n represents 0):

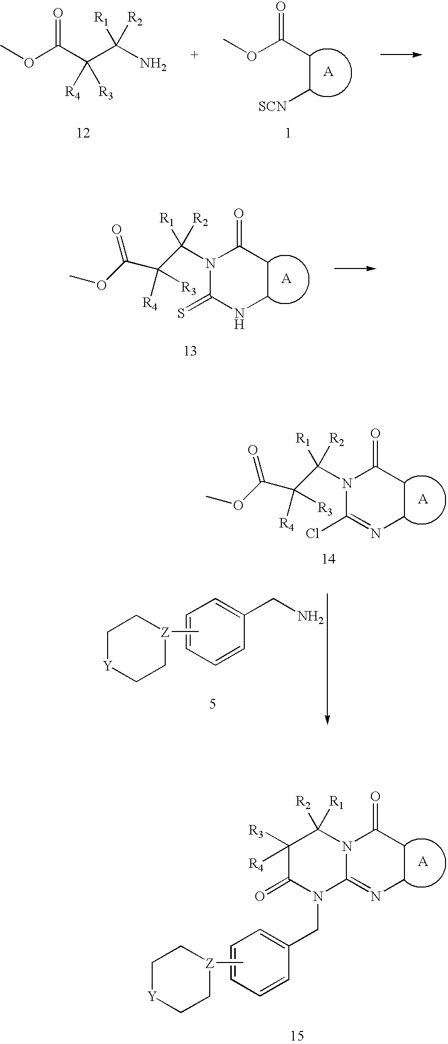

As described in Diagram C, the n-amino-ester (12) can react with an isothiocyanate ortho-ester (1) in a polar solvent such as methanol in the presence of a tertiary amine such as triethylamine at a temperature of 18 to 50° C., for 3 to 96 hours in order to produce the thioxo-pyrimidinone derivative (13). The derivative (13) can be chlorinated by reaction with phosphorus oxychloride in the presence or absence of phosphorus pentachloride, at ambient temperature or heated by microwaves to a temperature of 100 to 150° C. The chlorinated derivative (14) can react with the amine (5) in an aprotic solvent such as tetrahydrofuran or dimethylformamide, in the presence of an inorganic base or tertiary amine such as triethylamine, at a temperature of 80 to 120° C. in order to produce the pyrimido-pyrimidine-dione derivative (15).

### EXAMPLE C1

### 4,4-dimethyl-1-[4-(4-methylpiperazin-1-yl)benzyl]-3,4-dihydro-2H-pyrimido[2,1-b]quinazoline-2,6(1H)-dione hydrochloride

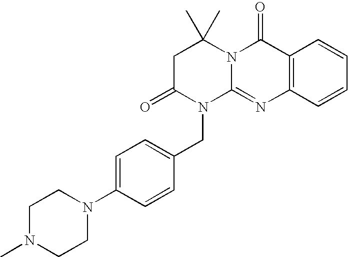

### Stage 1: methyl 3-methyl-3-(4-oxo-2-thioxo-1,4-dihydroquinazolin-3(2H)-yl)butanoate

Thionyl chloride (1.56 mL) is added to a solution cooled down to 0° C., of 3-amino-3-methylbutanoic acid (1 g) in methanol (8 mL). The mixture is stirred for 10 minutes at 0° C. then brought to ambient temperature and heated under reflux for 3 hours. The mixture is then concentrated under reduced pressure at 40° C. then dichloromethane is added to the oily residue and the reaction medium is again concentrated under reduced pressure at 40° C. This co-evaporation is repeated several times until methyl 3-amino-3-methylbutanoate is obtained in solid form (1.83 g, quantitative yield). Triethylamine (10 mL) then a solution of methyl 2-isothiocyanatobenzoate (1.65 g) in methanol (2 mL) are added to a solution of methyl 3-amino-3-methylbutanoate (1.43 g) in methanol (4 mL). The mixture is stirred at ambient temperature for 4 days then filtered. The solid obtained is washed with a minimum amount of methanol then with diethyl ether then dried in order to produce the expected compound in the form of a white powder (1.2 g, 48% yield).

MS/LC: calculated MM=292.1; m/z=293.1 (MH+)

NMR (¹H, 400 MHz, DMSO-d₆): δ1.36 (s, 3H), 1.40 (s, 3H), 2.76 (dd, 2H), 3.75 (s, 3H), 7.23 (AB, 1H), 7.48 (t, 1H), 7.63 (t, 1H), 7.94 (AB, 1H), 9.98 (s, 1H).

### Stage 2: methyl 3-(2-chloro-4-oxoquinazolin-3(4H)-yl)-3-methylbutanoate

Phosphorus pentachloride (200 mg) is added to a suspension of methyl 3-methyl-3-(4-oxo-2-thioxo-1,4-dihydroquinazolin-3(2H)-yl)butanoate (146 mg) in phosphorus oxychloride (2 mL). The mixture is stirred for 18 hours at ambient temperature then concentrated under reduced pressure at 40° C. Dichloromethane and water are added to the residue. After decantation and extractions, the combined organic phases are washed with salt water, dried over Na₂SO₄ then concentrated under reduced pressure at 40° C. in order to yield the expected product in the form of a colourless oil, used in the following stage without subsequent purification.

### Stage 3: 4,4-dimethyl-1-[4-(4-methylpiperazin-1-yl)benzyl]-3,4-dihydro-2H-pyrimido[2,1-b]quinazoline-2,6(1H)-dione hydrochloride

[4-(4-methylpiperazin-1-yl)benzyl]amine (205 mg) then potassium carbonate (170 mg) are added to methyl 3-(2-chloro-4-oxoquinazolin-3(4H)-yl)-3-methylbutanoate (146 mg) in anhydrous DMF (5 mL). The mixture is heated at 120° C. for 6 hours then concentrated under reduced pressure at 40° C. Dichloromethane and water are added to the residue. After decantation and extractions, the combined organic phases are washed with salt water, dried over Na₂SO₄ then concentrated under reduced pressure at 40° C. Purification by flash chromatography on silica gel (eluent: dichloromethane/methanol 95:5) produces the expected compound in the form of the free base. The corresponding hydrochloride salt is formed by adding a 1N HCl solution in ethyl ether to the solution of the free base in ethyl acetate. The precipitate obtained is filtered and dried in order to produce the expected hydrochloride compound (50 mg, 20% yield from Stage 2).

MS/LC: calculated MM=431.2; m/z=432.3 (MH+)

NMR (¹H, 400 MHz, DMSO-d₆): δ1.63 (s, 6H), 2.77 (d, 3H), 2.98 (m, 4H), 3.11 (m, 2H), 3.42 (d, 2H), 3.74 (d, 2H), 5.17 (s, 2H), 6.90 (AB, 1H), 7.25 (AB, 1H), 7.38 (t, 1H), 7.48 (AB, 1H), 7.74 (t, 1H), 8.0 (AB, 1H), 10.43 (s, 1H).

In a fashion analogous to the procedure described for 4,4-dimethyl-1-[4-(4-methylpiperazin-1-yl)benzyl]-3,4-dihydro-2H-pyrimido[2,1-b]quinazoline-2,6(1H)-dione hydrochloride, 5-[4-(4-methylpiperazin-1-yl)benzyl]-7,8-dihydro-6H-pyrimido[1,2-c]thieno[3,2-d]pyrimidine-6,10(5H)-dione hydrochloride as well as the following compounds were prepared:

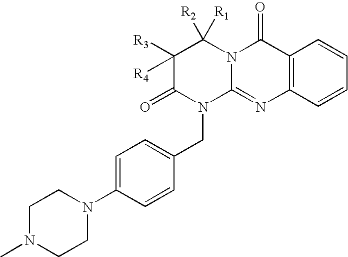

in which R₂, R₃ and R₄ represent the hydrogen atom and R₁ represents one of the radicals below:

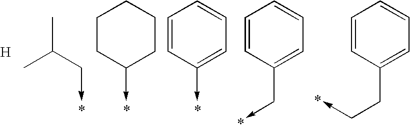

in which R₁, R₂ and R₃ represent the hydrogen atom and R₄ represents the benzyl radical;
in which R₁, R₂, R₃ and R₄ represent the hydrogen atom; and
in which R₁ and R₂ represent the hydrogen atom and R₃ and R₄ represent the methyl radical.

### EXAMPLE C2

### 6-[4-(4-methylpiperazin-1-yl)benzyl]-5H-quinazolino[3,2-a]quinazoline-5,12(6H)-dione hydrochloride

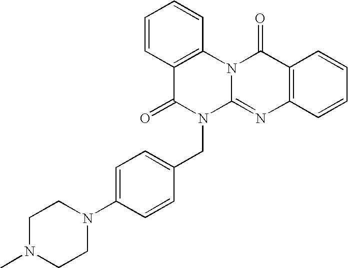

### Stage 1: methyl 2-(4-oxo-2-thioxo-1,4-dihydroquinazolin-3(2H)-yl)benzoate

A mixture of methyl 2-aminobenzoate (0.76 g) and methyl 2-isothiocyanatobenzoate (1.1 g) in methanol (2 mL) and triethylamine (2 mL) is stirred at ambient temperature for 24 hours. The precipitate formed is filtered and washed with methanol then ethyl ether then dried in order to produce the expected compound in the form of a white powder (0.6 g, 36% yield).

MS/LC: calculated MM=312.3; m/z=313.2 (MH+)

NMR (¹H, 400 MHz, DMSO-d₆): δ3.65 (s, 3H), 7.31-8.07 (m, 8H), 13.05 (s, 1H).

### Stage 2: methyl 2-(2-chloro-4-oxoquinazolin-3(4H)-yl)benzoate

Phosphorus pentachloride (200 mg) is added to a suspension of methyl 2-(4-oxo-2-thioxo-1,4-dihydroquinazolin-3(2H)-yl)benzoate (160 mg) in phosphorus oxychloride (2 mL). The mixture is stirred for 18 hours at ambient temperature then concentrated under reduced pressure at 40° C. Dichloromethane and water are added to the residue. After decantation and extractions, the combined organic phases are washed with salt water, dried over Na₂SO₄ then concentrated under reduced pressure at 40° C. in order to yield the expected product in the form of a colourless oil, used in the following stage without subsequent purification.

MS/LC: calculated MM=314.1; m/z=315.2 (MH+)

### Stage 3: 6-[4-(4-methylpiperazin-1-yl)benzyl]-5H-quinazolino[3,2-a]quinazoline-5,12(61)-dione hydrochloride

A solution of methyl 2-(2-chloro-4-oxoquinazolin-3(4H)-yl)benzoate (170 mg) and [4-(4-methylpiperazin-1-yl)benzyl]amine (200 mg) in a THF/DCM/Et₃N mixture (1 mL/1 mL/1 mL) is stirred for 4 days at ambient temperature then water and dichloromethane are added. After decantation and extractions, the combined organic phases are washed with salt water, dried over Na₂SO₄ then concentrated under reduced pressure at 40° C. Purification by flash chromatography on silica gel (eluent: dichloromethane/methanol 100:0 to 92:8) produces the expected compound in the form of the free base. The corresponding hydrochloride salt is formed by adding a 1N HCl solution in ethyl ether to the solution of the free base in ethyl acetate. The precipitate obtained is filtered and dried in order to produce the expected hydrochloride compound (85 mg, 36% yield from Stage 2).

MS/LC: calculated MM=431.2; m/z=432.3 (MH+)

NMR (¹H, 400 MHz, DMSO-d₆): δ2.83 (d, 3H), 3.12 (m, 4H), 3.48 (d, 2H), 3.81 (d, 2H), 5.55 (s, 2H), 6.98 (AB, 2H), 7.55 (m, 3H), 7.66 (m, 2H), 7.90 (m, 2H), 8.25 (AB, 1H), 8.31 (AB, 1H), 9.11 (AB, 1H), 10.75 (s, 1H).

Preparation according to reaction diagram D (case where m and n represent 1):

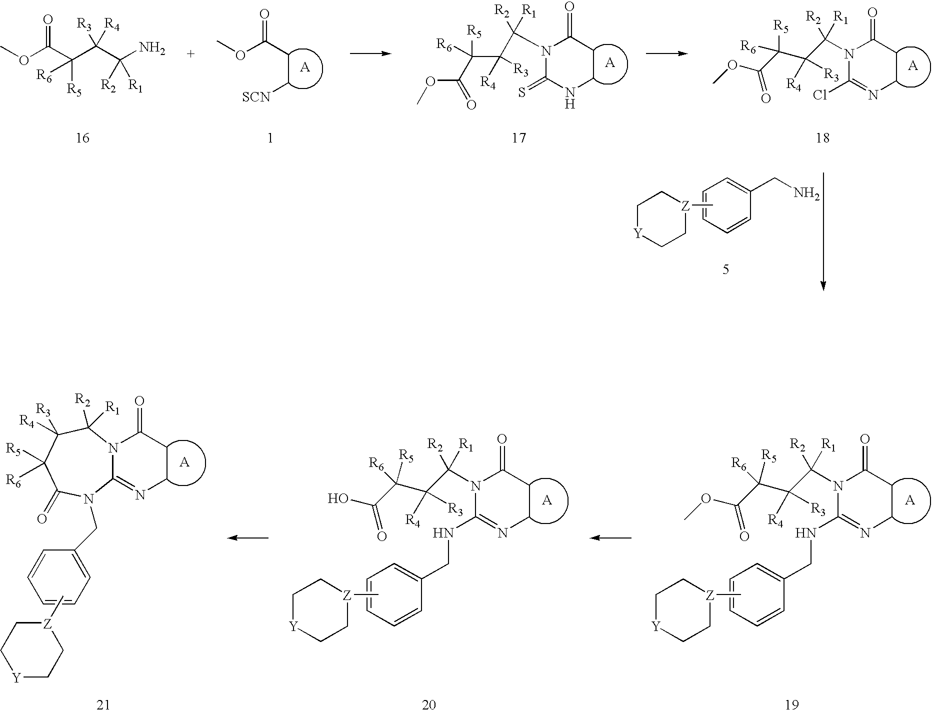

As described in Diagram D, the γ-amino-ester (16) can react with an ortho-ester isothiocyanate (1) in a polar solvent such as methanol in the presence of a tertiary amine such as triethylamine at a temperature of 18 to 50° C., for 3 to 96 hours in order to produce the thioxo-pyrimidinone derivative (17). The derivative (17) can be chlorinated by reaction with phosphorus oxychloride in the presence or absence of phosphorus pentachloride, at ambient temperature or heated by microwaves to a temperature of 100 to 150° C. in order to produce the derivative (18). The chlorinated derivative 18 can react with the amine (5) in an aprotic solvent such as tetrahydrofuran in the presence or absence of an organic base such as triethylamine, at ambient temperature or heated by microwaves to a temperature of 80 to 120° C. in order to produce the derivative (19). The ester (19) is then saponified in the presence of a base such as soda, potash, potassium tertbutylate or lithium hydroxide in an aprotic solvent such as tetrahydrofuran or in a protic solvent such as methanol, at a temperature of 18 to 80° C., for 2 to 24 hours in order to produce the corresponding acid (20). The derivative (20) can be treated with a coupling agent such as diisopropylcarbodiimide (DIC), dicyclohexylcarbodiimide (DCC), 1-(3-dimethylaminopropyl)-3-ethylcarbodiimide hydrochloride (EDC) or carbonyldiimidazole (CDI), with or without 1-hydroxybenzotriazole (HOBt), or with Mukaiyama's reagent (2-chloro-1-methyl-pyridinium chloride) in the presence of a tertiary amine such as triethylamine or diisopropylethylamine, in an inert organic solvent such as methylene chloride, tetrahydrofuran or dimethylformamide at ambient temperature for 3 to 24 hours or alternatively heated by microwaves to a temperature of 80 to 120° C. (Biotage® equipment), in a sealed tube, for 10 to 30 minutes, in order to produce the derivative (21).

### EXAMPLE D1

### 1-[4-(4-methylpiperazin-1-yl)benzyl]-4,5-dihydro[1,3]diazepino[2,1-b]quinazoline-2,7(1H,3H)-dione hydrochloride

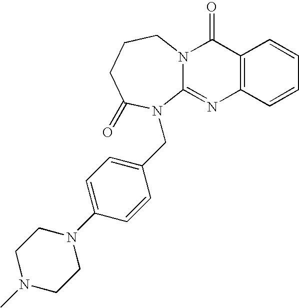

### Stage 1: methyl 4-(4-oxo-2-thioxo-1,4-dihydroquinazolin-3(2H)-yl)butanoate

Triethylamine (5 mL) then methyl 2-isothiocyanatobenzoate (1.65 g) are added to a solution of methyl 4-aminobutanoate (2 g) in methanol (5 mL). The mixture is stirred at ambient temperature for 24 hours, then filtered. The solid obtained is washed with a minimum amount of methanol then with diethyl ether then dried in order to produce the expected compound in the form of a white powder (3.1 g, 86% yield).

MS/LC: calculated MM=278.1; m/z=279.1 (MH+)

NMR (¹H, 400 MHz, DMSO-d₆): δ1.96 (t, 2H), 2.38 (t, 2H), 3.54 (s, 3H), 4.43 (t, 2H), 7.31 (t, 1H), 7.36 (AB, 1H), 7.71 (t, 1H), 7.95 (AB, 1H), 12.88 (s, 1H).

### Stage 2: methyl 4-(2-chloro-4-oxoquinazolin-3(4H)-yl)butanoate

Phosphorus pentachloride (200 mg) is added to a suspension of methyl 4-(4-oxo-2-thioxo-1,4-dihydroquinazolin-3(2H)-yl)butanoate (139 mg) in phosphorus oxychloride (2 mL). The mixture is stirred for 18 hours at ambient temperature then concentrated under reduced pressure at 40° C. Dichloromethane and water are added to the residue. After decantation and extractions, the combined organic phases are washed with salt water, dried over Na₂SO₄ then concentrated under reduced pressure at 40° C. in order to yield the expected product in the form of a colourless oil, used in the following stage without subsequent purification.

MS/LC: calculated MM=280.1; m/z=281.1 (MH+)

### Stage 3: methyl 4-[2-[4-(4-methylpiperazin-1-yl)benzyl]amino-4-oxoquinazolin-3(4H)-yl]butanoate

Triethylamine (0.4 mL) and [4-(4-methylpiperazin-1-yl)benzyl]amine (300 mg) are successively added to a solution of methyl 4-(2-chloro-4-oxoquinazolin-3(4H)-yl)butanoate (140 mg) in tetrahydrofuran (3 mL). The mixture is stirred at ambient temperature for 24 hours then water and ethyl acetate are added. After decantation and extractions, the combined organic phases are washed with salt water, dried over Na₂SO₄ then concentrated under reduced pressure at 40° C. Purification by flash chromatography on silica gel (eluent: dichloromethane/methanol 95:5) produces the expected compound in the form of beige crystals (131 mg, 58% yield from Stage 1).

MS/LC: calculated MM=449.2; m/z=450.2 (MH+)

NMR (¹H, 400 MHz, DMSO-d₆): δ 1.85 (t, 2H), 2.21 (s, 3H), 2.40 (m, 6H), 3.08 (m, 4H), 3.54 (s, 3H), 4.05 (t, 2H), 4.54 (d, 2H), 6.86 (AB, 2H), 7.08 (t, 2H), 7.18 (AB, 1H), 7.25 (AB, 2H), 7.48 (t, 1H), 7.52 (t, 1H), 7.89 (AB, 1H).

### Stage 4: 4-[2-{[4-(4-methylpiperazin-1-yl)benzyl]amino}-4-oxoquinazolin-3(4H)-yl]butanoic acid

A solution of methyl 4-[2-{[4-(4-methylpiperazin-1-yl)benzyl]amino}-4-oxoquinazolin-3(4H)-yl]butanoate (100 mg) in tetrahydrofuran (2 mL) is added to a suspension of potassium tertbutylate (50 mg) in tetrahydrofuran (2 mL). The mixture is stirred for 2 hours at ambient temperature then concentrated under reduced pressure at 40° C. Dichloromethane and water are added to the residue. After decantation, the aqueous phase is acidified with acetic acid then extracted several times with dichloromethane. The combined organic phases are washed with salt water, dried over Na₂SO₄ then concentrated under reduced pressure at 40° C. The acid thus obtained is dried and used in the following stage without subsequent purification (77 mg, 81% yield).

MS/LC: calculated MM=435.2; m/z=4363 (MH+)

### Stage 5: 1-[4-(4-methylpiperazin-1-yl)benzyl]-4,5-dihydro[1,3]diazepino[2,1-b]quinazoline-2,7(1H,3H)-dione hydrochloride

1-(3-dimethylaminopropyl)-3-ethylcarbodiimide hydrochloride (EDC) (62 mg), 1-hydroxybenzotriazole (HOBt) (44 mg) and diisopropylethylamine (115 μμL) are successively added to a solution of 4-[2-{[4-(4-methylpiperazin-1-yl)benzyl]amino}-4-oxoquinazolin-3(4H)-yl]butanoic acid (70 mg) in dichloromethane (3 mL). The mixture is stirred at ambient temperature for 24 hours then water and dichloromethane are added to it. After decantation and extractions, the combined organic phases are washed with salt water, dried over Na₂SO₄ then concentrated under reduced pressure at 40° C. Purification by flash chromatography on silica gel (eluent: dichloromethane/methanol 95:5) produces the expected compound in the form of the free base. The corresponding hydrochloride salt is formed by adding a 1N HCl solution in ethyl ether to the solution of the free base in ethyl acetate. The precipitate obtained is filtered and dried in order to produce the expected hydrochloride compound (21 mg, 30% yield).

MS/LC: calculated MM=417.2; m/z=418.3 (MH+)

NMR (¹H, 400 MHz, CDCl₃): δ2.19 (t, 2H), 2.65 (m, 2H), 2.85 (d, 3H), 3.02 (m, 2H), 3.12 (m, 2H), 3.50 (d, 2H), 3.83 (d, 2H), 4.20 (m, 2H), 5.12 (s, 2H), 6.96 (AB, 2H), 7.31 (AB, 2H), 7.57 (m, 1H), 7.70 (AB, 1H), 7.89 (m, 1H), 8.16 (AB, 1H), 10.5 (s, 1H).

In a fashion analogous to the procedure described for 1-[4-(4-methylpiperazin-1-yl)benzyl]-4,5-dihydro[1,3]diazepino[2,1-b]quinazoline-2,7(1H,3H)-dione hydrochloride, 1′-[4-(4-methylpiperazin-1-yl)benzyl]spiro[cyclohexane-1,4′-[1,3]diazepino[2,1-b]quinazoline]-2′,7′(1′H,3′H)-dione hydrochloride was prepared.

Preparation according to reaction diagram E (case where Z represents a nitrogen atom and Y represents —NR₈):

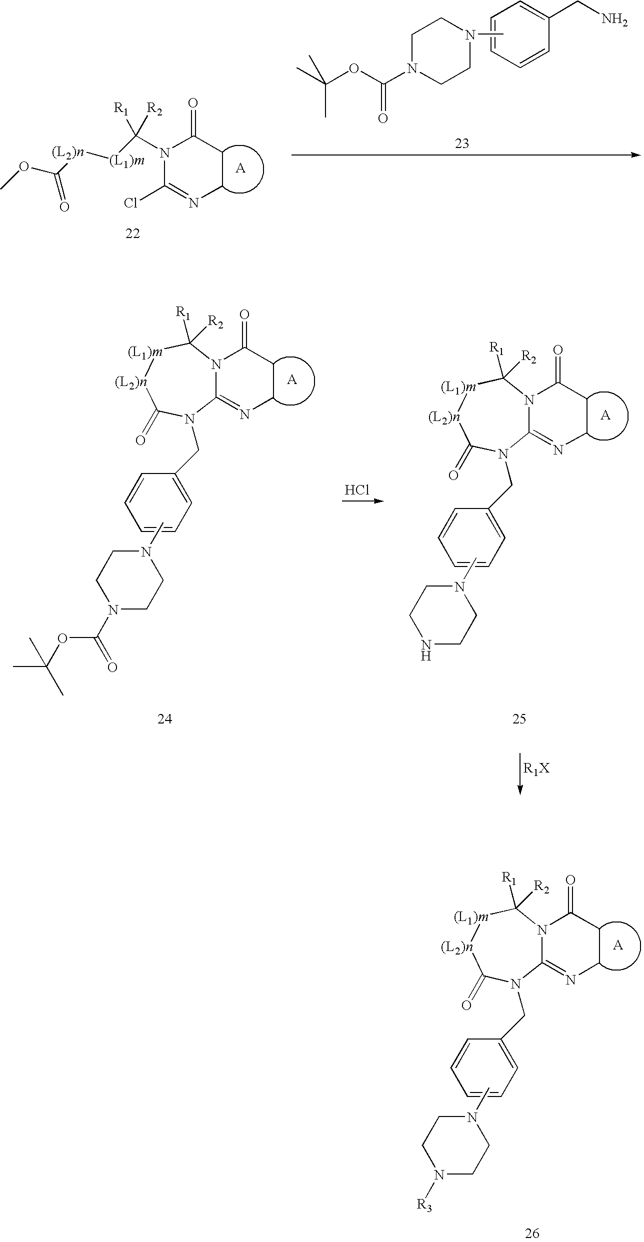

As described in Diagram E, the chlorinated derivative (22) can react with the amine (23) in an aprotic solvent such as tetrahydrofuran in the presence or absence of an inorganic base or tertiary amine such as triethylamine, at ambient temperature or heated by microwaves to a temperature of 80 to 120° C. in order to produce the derivative (24). The N-Boc derivative (24) can be deprotected by acid treatment such as hydrogen chloride or trifluoroacetic acid in an aprotic solvent such as dichloromethane or dioxane for 1 to 4 hours, at ambient temperature, in order to produce the derivative (25). The piperazine (25) can be alkylated with a halogenated reagent in the presence of an inorganic base or tertiary amine such as triethylamine in an aprotic solvent such as tetrahydrofuran, at ambient temperature or alternatively can be treated with an aldehyde followed by a reduction of the imine by a reducing agent such as sodium cyanoborohydride, in order to produce the derivative (26).

### EXAMPLE E1

### 1-[4-(4-ethylpiperazin-1-yl)benzyl]-3-isobutylimidazo[2,1-b]quinazoline-2,5(1H,3H)-dione hydrochloride

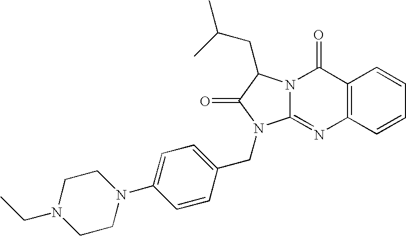

### Stage 1: tert-butyl 4-{4-[(3-isobutyl-2,5-dioxo-2,3-dihydroimidazo[2,1-b]quinazolin-1(5H)-yl)methyl]phenyl}piperazine-1-carboxylate

Tert-butyl 4-[4-(aminomethyl)phenyl]piperazine-1-carboxylate (930 mg) and triethylamine (0.56 mL) are added to a solution of methyl 1-(2-chloro-4-oxoquinazolin-3(4H)-yl)cyclopentane carboxylate (prepared according to Example A2; 620 mg) in THF (5 mL). The mixture is stirred for 48 hours at ambient temperature then the precipitate formed is filtered and washed with THF. The filtrate is concentrated under reduced pressure at 40° C. then purified by flash chromatography on silica gel (eluent: 100% heptane to heptane/ethyl acetate 7:3) produces the expected compound in the form of white powder (880 mg, 83% yield).

MS/LC: calculated MM=531.3; m/z=532.3 (MH+)

NMR (¹H, 400 MHz, DMSO-d₆): δ0.80 (m, 6H), 1.40 (s, 9H), 1.79 (m, 1H), 2.01 (m, 2H), 3.05 (t, 4H), 3.41 (t, 4H), 4.77 (AB, 1H), 4.82 (AB, 1H), 4.93 (t, 1H), 6.90 (AB, 2H), 7.30 (AB, 2H), 7.41 (t, 1H), 7.57 (AB, 1H), 7.77 (t, 1H), 8.08 (AB, 1H).

### Stage 2: 3-isobutyl-1-(4-piperazin-1-ylbenzyl)imidazo[2,1-b]quinazoline-2,5(1H,3H)-dione hydrochloride

A solution of hydrochloric acid in dioxane (4N, 4 mL) is added to tert-butyl 4-{4-[(3-isobutyl-2,5-dioxo-2,3-dihydroimidazo[2,1-b]quinazolin-1(5H)-yl)methyl]phenyl}piperazine-1-carboxylate (880 mg). The solution is stirred for 4 hours at ambient temperature then concentrated under reduced pressure at 40° C. The powder obtained is dried (720 mg, 90% yield).

MS/LC: calculated MM=431.2; m/z=432.3 (MH+)

NMR (¹H, 400 MHz, DMSO-d₆): δ0.80 (m, 6H), 1.79 (m, 1H), 2.01 (m, 2H), 3.16 (m, 4H), 3.33 (m, 4H), 4.78 (AB, 1H), 4.83 (AB, 1H), 4.93 (t, 2H), 6.95 (AB, 2H), 7.33 (AB, 2H), 7.40 (t, 1H), 7.57 (AB, 1H), 7.76 (t, 1H), 8.08 (AB, 1H), 9.28 (s, 2H).

### Stage 3: 1-[4-(4-ethylpiperazin-1-yl)benzyl]-3-isobutylimidazo[2,1-b]quinazoline-2,5(1H,3H)-dione hydrochloride

Iodoethane (19 mg) is added to a suspension of 3-isobutyl-1-(4-piperazin-1-ylbenzyl)imidazo[2,1-b]quinazoline-2,5(1H,3H)-dione hydrochloride (56 mg) and sodium carbonate (40 mg) in anhydrous acetonitrile (1 mL), placed in a “Biotage®” reaction tube. The reaction tube is sealed with a cap, placed in a “Biotage®” micro-wave and heated under magnetic stirring at 100° C. for 1 hour. The mixture is concentrated under reduced pressure at 40° C. then water and dichloromethane are added. After decantation and extractions, the combined organic phases are washed with salt water, dried over Na₂SO₄ then concentrated under reduced pressure at 40° C. Purification of the residue obtained by flash chromatography on silica gel (eluent: 100% dichloromethane to dichloromethane/ethyl methanol 95:5) produces the expected compound in the form of the free base. The corresponding hydrochloride salt is formed by adding a 1N HCl solution in ethyl ether to the solution of the free base in ethyl acetate. The precipitate obtained is filtered and dried in order to produce the expected hydrochloride compound (64 mg, 65% yield).

MS/LC: calculated MM=459.3; m/z=460.3 (MH+)

NMR (¹H, 400 MHz, DMSO-d₆): δ0.80 (m, 6H), 1.26 (t, 3H), 1.79 (m, 1H), 2.00 (m, 2H), 3.10 (m, 6H), 3.50 (m, 2H), 3.78 (m, 2H), 4.80 (AB, 1H), 4.88 (AB, 1H), 4.94 (t, 2H), 6.96 (AB, 2H), 7.33 (AB, 2H), 7.41 (t, 1H), 7.57 (AB, 1H), 7.76 (t, 1H), 8.08 (AB, 1H), 10.92 (s, 1H).

In a fashion analogous to the procedure described for 1-[4-(4-ethylpiperazin-1-yl)benzyl]-3-isobutylimidazo[2,1-b]quinazoline-2,5(1H,3H)-dione hydrochloride, the following compounds were prepared:

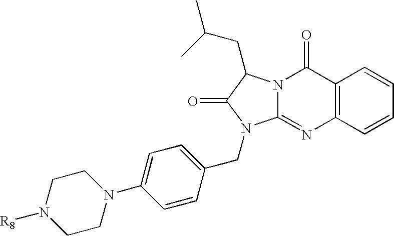

in which R₈ represents one of the radicals below:

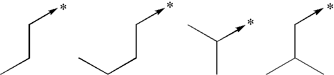

A subject of the invention is also a method for preparing a compound of formula (I) as defined above, characterized in that the chlorinated derivative (II)

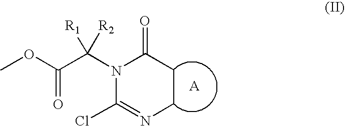

in which A, R₁ and R₂ are as defined above, is reacted with the amine (III)

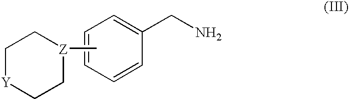

in which Y and Z are as defined above, in an aprotic solvent in the presence or absence of an inorganic base or tertiary amine, at a temperature of 18-80° C., in order to produce compound (I) in which n and m represent 0.

A subject of the invention is also a method for preparing a compound of formula (I) as defined above, characterized in that the chlorinated derivative (IV)

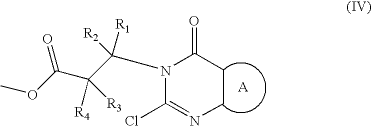

in which A, R₁, R₂, R₃ and R₄ are as defined above, is reacted with the amine (III)

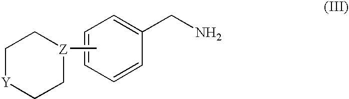

in which Y and Z are as defined above, in an aprotic solvent in the presence of an inorganic base or tertiary amine, at a temperature of 80 to 120° C., in order to produce compound (I) in which n and m represent 1 and 0 respectively.

A subject of the invention is also a method for preparing a compound of formula (I) as defined above, characterized in that the chlorinated derivative (V)

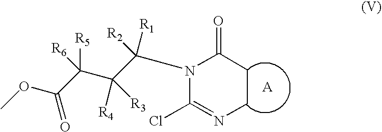

in which A, R₁, R₂, R₃, R₄, R₅ and R₆ are as defined above, is reacted with the amine (III)

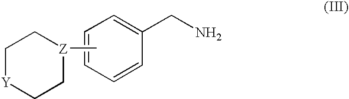

in which Y and Z are as defined above, in an aprotic solvent in the presence or absence of an organic base, in order to form the ester (VI)

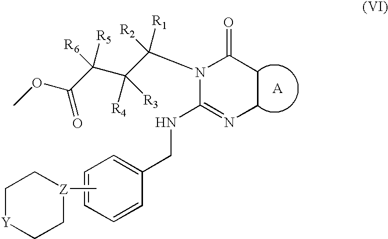

the ester (VI) thus formed is saponified in the presence of a base in an aprotic or protic solvent, at a temperature of 18 to 80° C., for 2 to 24 hours, in order to produce the corresponding acid (VII)

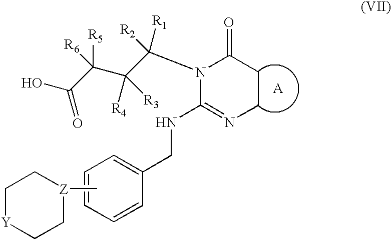

and the derivative (VII) is treated with a coupling agent or with Mukaiyama's reagent in the presence of a tertiary amine, in an inert organic solvent, at ambient temperature for 3 to 24 hours, in order to produce compound (I) in which n and m represent 1.

The compounds of the present invention possess useful pharmacological properties. It was thus discovered that the compounds of the present invention possess a good affinity for certain sub-types of cannabinoid receptors, in particular CB2 receptors. They are particularly useful for treating the pathological states and diseases in which one or more cannabinoid receptors are involved.

The compounds of the present invention can thus be used in different therapeutic applications. They can advantageously be used for the treatment and prevention of the pathological states and diseases associated with the activity of the cannabinoid receptors such as cell proliferation disorders such as cancer, immune disorders, inflammation, pain, osteoporosis, atherosclerosis, epilepsy, nausea associated with chemotherapy treatments, fibrosis, gastro-intestinal disorders, neurodegenerative diseases including multiple sclerosis and dyskinesia, Parkinson's disease, Huntington's chorea and Alzheimer's disease. They can also be used in order to prevent or cure diseases associated with motor function such as Tourette's syndrome, or in order to provide neuroprotection. The compounds according to the present invention can be administered alone or in combination with other agents linked to treatments of the symptoms or the cause of the disease or pathological state as mentioned above. An illustration of the pharmacological properties of the compounds of the invention will be found hereafter, in the experimental part.

A subject of the present application is also pharmaceutical compositions containing, as active ingredient, at least one product of formula I as defined above, or an addition salt with the pharmaceutically acceptable mineral or organic acids of said product of formula I, in combination with a pharmaceutically acceptable support.

A subject of the present application is also the use of the compounds according to the present invention, for the preparation of a medicament for the treatment of cell proliferation disorders and preferably for the treatment of cancer.

A subject of the present application is also the use of the compounds according to the present invention, for the preparation of a medicament for the treatment of immune disorders, inflammation, pain, osteoporosis, neurodegenerative diseases including multiple sclerosis and dyskinesia, and Parkinson's disease.

The pharmaceutical composition can be in the form of a solid, for example, powders, granules, tablets, gelatin capsules or suppositories. Appropriate solid supports can be, for example, calcium phosphate, magnesium stearate, talc, sugars, lactose, dextrin, starch, gelatin, cellulose, methyl cellulose, sodium carboxymethyl cellulose, polyvinylpyrrolidine and wax.

The pharmaceutical compositions containing a compound of the invention can also be presented in liquid form, for example, solutions, emulsions, suspensions or syrups. Appropriate liquid supports can be, for example, water, organic solvents such as glycerol or the glycols, as well as their mixtures, in varying proportions, in water, added to pharmaceutically acceptable oils or fats. The sterile liquid compositions can be used for intramuscular, intraperitoneal or sub-cutaneous injections and the sterile compositions can also be administered intravenously.

All the technical and scientific terms used in the present text have the meaning known to a person skilled in the art. Moreover, all the patents (or patent applications) as well as the other bibliographical references are incorporated by way of reference.

### Experimental Part:

The compounds according to the invention obtained according to the previously described procedures are shown in the table below.

The compounds are characterized by their retention time (rt) and their molecular peak determined by mass spectrometry (MH+).

For the mass spectrometry, a single quadripole mass spectrometer (Micromass, Platform model) equipped with an electrospray source is used with a resolution of 0.8 Da at 50% valley. Calibration is carried out monthly between the masses 80 and 1000 Da using a calibration mixture of sodium iodide and rubidium iodide in solution in an isopropanol/water mixture (1/1 Vol.).

For the liquid chromatography, a Waters system comprising an in-line degasser, a Waters 600 quaternary pump, a Gilson 233 plate sampling injector and a Waters PAD 996 UV detector is used.

The elution conditions used are as follows:

Eluent: A water+0.02% trifluoroacetic acid; B acetonitrile

| T (min) | A % | B % |
|---|---|---|
| 1 | 95 | 5 |
| 8.5 | 5 | 95 |
| 10.5 | 5 | 95 |
| 10.6 | 95 | 5 |
| 14.9 | 95 | 5 |
| 15.0 | 95 | 5 |

Flow rate: 1 ml/min; Injection: 10 μl; Column: Uptisphere ODS 3 μm 75*4.6 mm i.d.

These examples are presented in order to illustrate the above procedures and should in no event be considered as limiting the scope of the invention.

| Examples | Molecular structures | [M + H]+ | rt (min) |
|---|---|---|---|
| 1 | 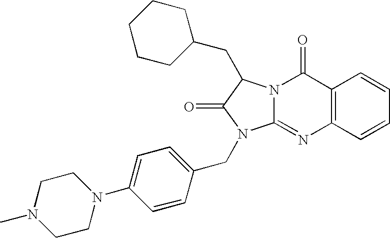 | 486.4 | 8.3 |
| 2 | 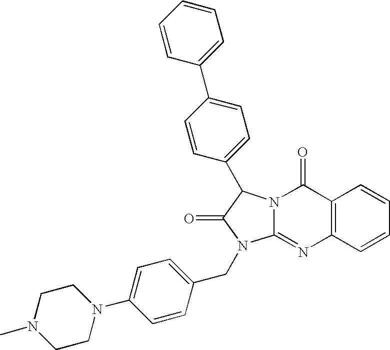 | 542.3 | 8.3 |
| 3 | 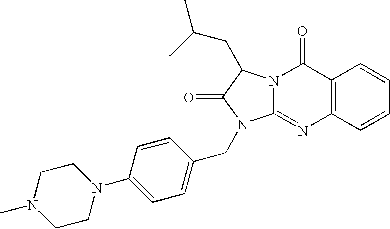 | 446.3 | 8.6 |
| 4 | 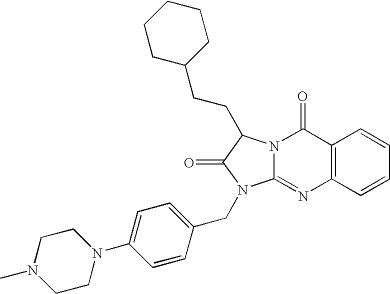 | 500.4 | 8.5 |
| 5 | 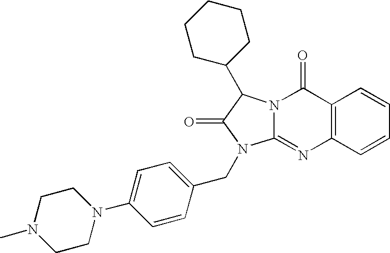 | 472.4 | 8.2 |
| 6 | 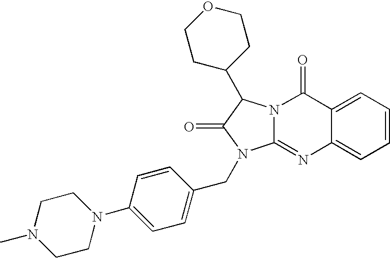 | 473.9 | 8.1 |
| 7 | 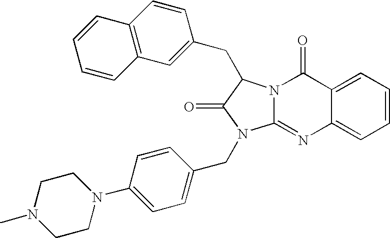 | 529.8 | 8.8 |
| 8 | 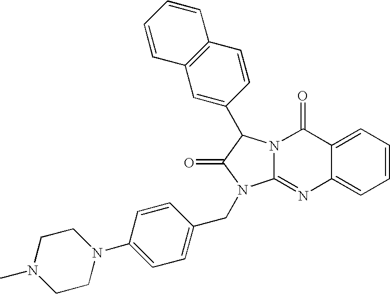 | 516.4 | 8.1 |
| 9 | 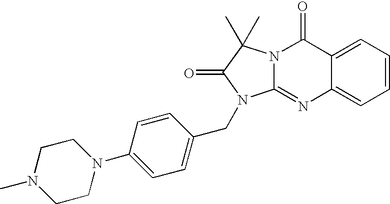 | 418.3 | 7.6 |
| 10 | 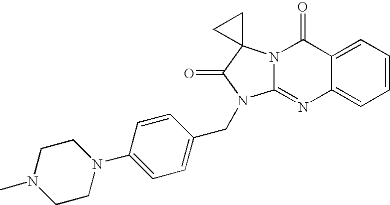 | 416.3 | 7.6 |
| 11 | 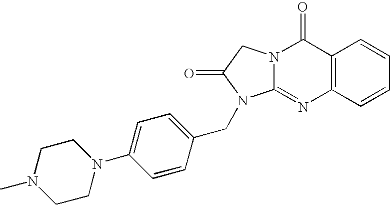 | 390.2 | 7.8 |
| 12 | 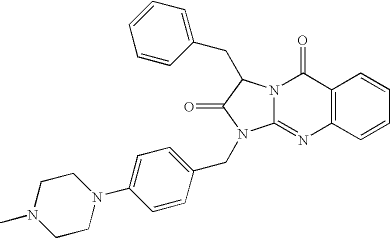 | 480.3 | 8.5 |
| 13 | 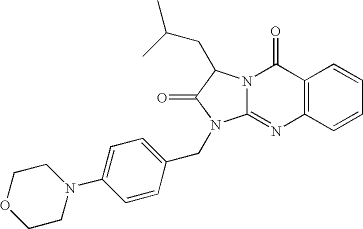 | 433.3 | 10.2 |
| 14 | 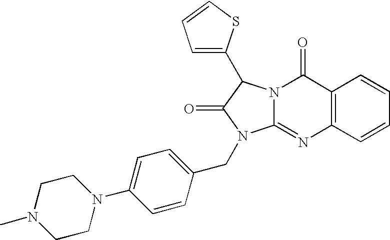 | 472.2 | 8.4 |
| 15 | 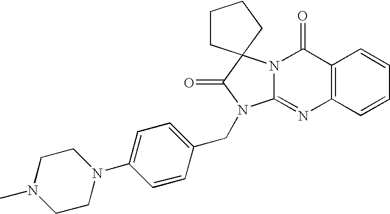 | 444.3 | 8.6 |
| 16 | 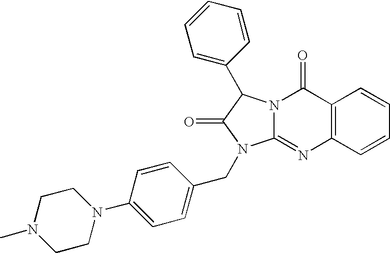 | 466.3 | 7.7 |
| 17 | 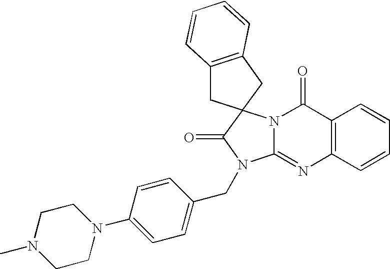 | 492.3 | 8.4 |
| 18 | 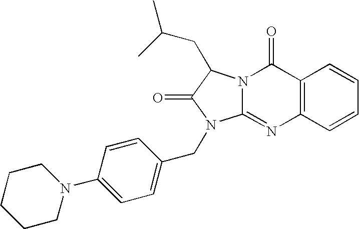 | 431.3 | 8.6 |
| 19 | 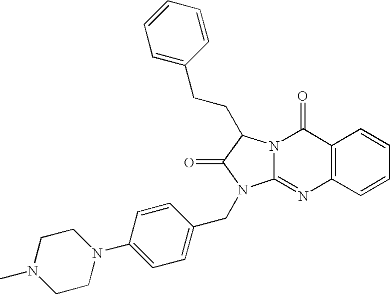 | 494.4 | 8.1 |
| 20 | 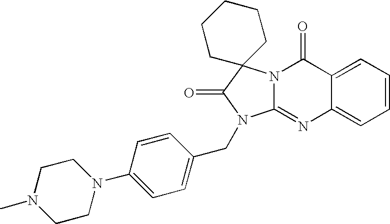 | 458.3 | 8.2 |
| 21 | 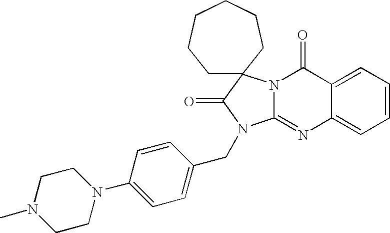 | 472.3 | 8.4 |
| 22 | 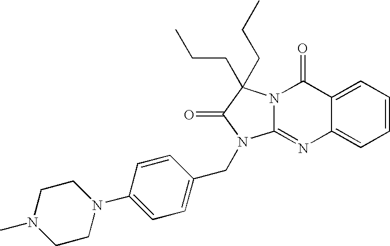 | 474.3 | 8.9 |
| 23 | 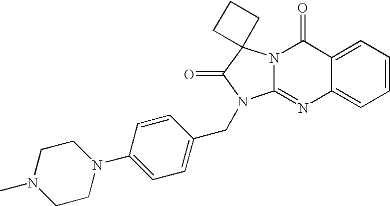 | 430.2 | 7.9 |
| 24 | 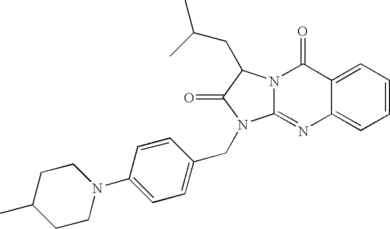 | 445.3 | 9.1 |
| 25 | 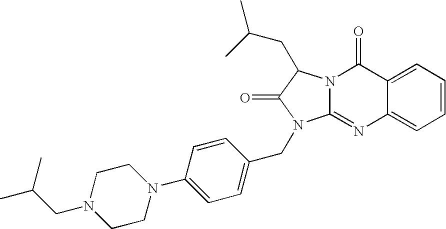 | 488.4 | 8.6 |
| 26 | 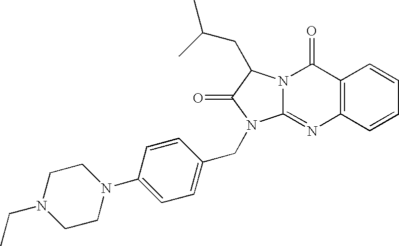 | 460.3 | 8.3 |
| 27 | 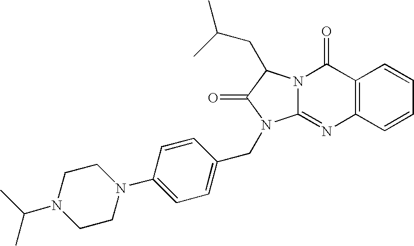 | 474.3 | 8.4 |
| 28 | 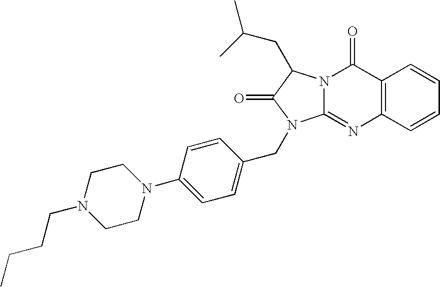 | 488.3 | 8.7 |
| 29 | 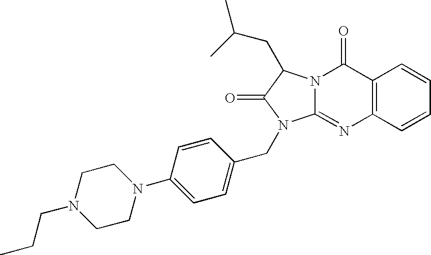 | 474.3 | 8.5 |
| 30 | 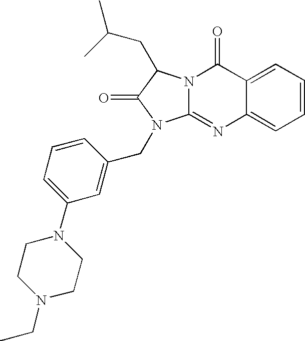 | 460.2 | 8.7 |
| 31 | 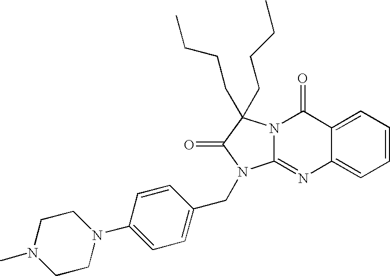 | 502.3 | 9.9 |
| 32 | 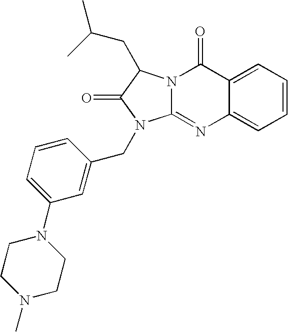 | 446.3 | 8.7 |
| 33 | 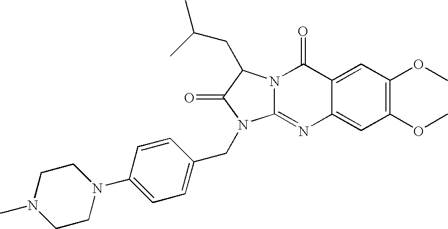 | 506.4 | 8.3 |
| 34 | 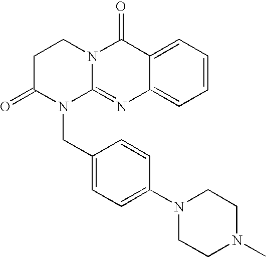 | 404.2 | 8.5 |
| 35 | 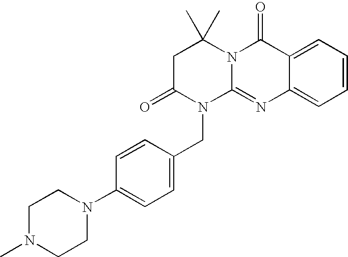 | 432.3 | 8.7 |
| 36 | 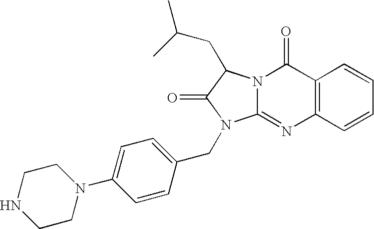 | 432.3 | 7.8 |
| 37 | 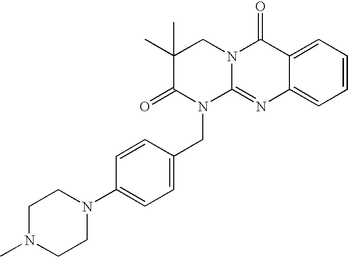 | 432.3 | 9.0 |
| 38 | 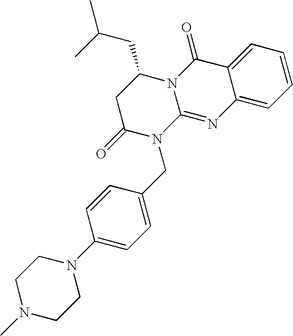 | 460.3 | 9.4 |
| 39 | 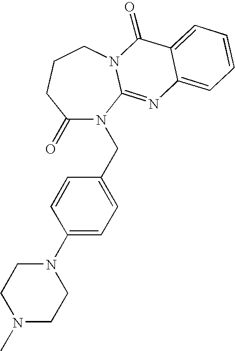 | 418.3 | 7.8 |
| 40 | 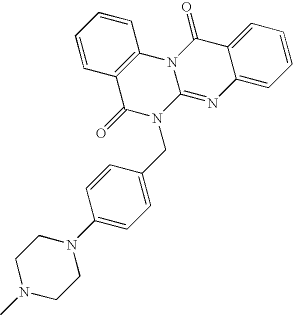 | 452.2 | 9.7 |
| 41 | 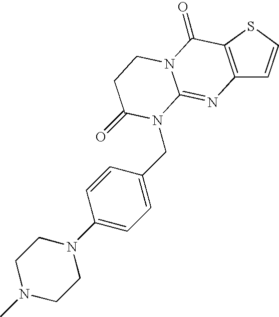 | 410.2 | 8.2 |
| 42 | 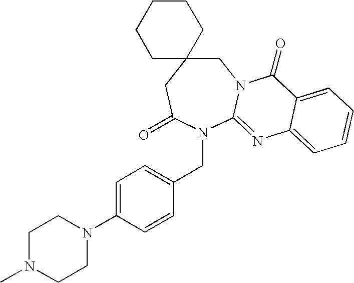 | 486.3 | 8.7 |
| 43 | 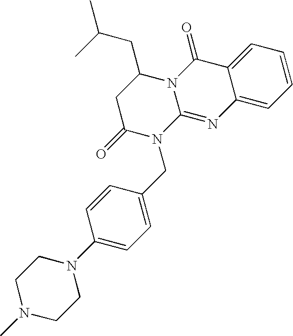 | 460.3 | 8.6 |
| 44 | 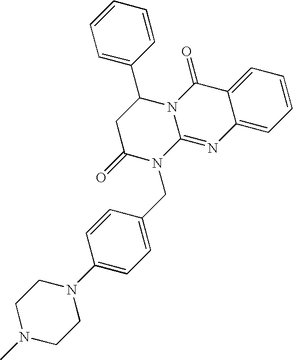 | 480.2 | 8.5 |
| 45 | 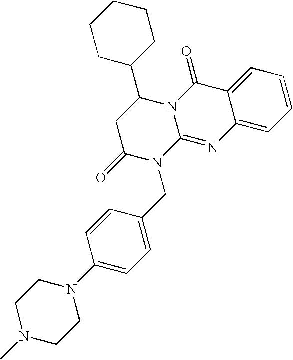 | 486.3 | 8.9 |
| 46 | 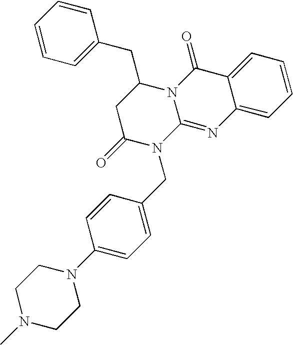 | 494.2 | 8.8 |
| 47 | 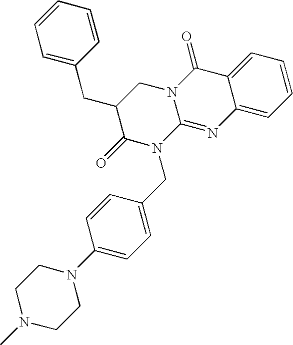 | 494.2 | 8.9 |
| 48 | 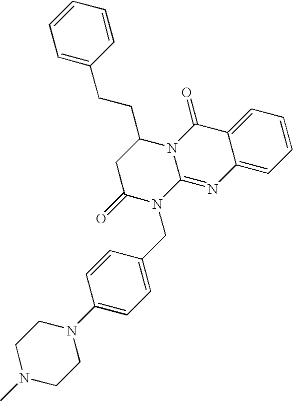 | 508.2 | 8.9 |

### Pharmacological Study

The affinity of the compounds of the present invention for the different sub-types of cannabinoid receptors was measured according to procedures analogous to those described hereafter for the human CB2 receptor.

### Study of the Affinity of the Compounds for the Human CB2 Cannabinoid Receptors

The affinity of the compounds of the invention for the human CB2 receptors is determined by measuring the inhibition of the binding of [³H]-CP55940 to transfected CHO-K1 cell membrane preparations.

The CHO-K1 cells expressing the human CB2 receptors in a stable fashion are cultured in an RPMI 1640 medium containing 10% foetal calf serum, 2 mM of glutamine, 100 U/ml of penicillin, 0.1 mg/ml of streptomycin and 0.5 mg/ml of G418. The cells are collected with 0.5 mM of EDTA and centrifuged at 500 g for 5 minutes at 4° C. The pellet is re-suspended in phosphate buffered saline medium (PBS) and centrifuged at 500 g for 5 minutes at 4° C. The pellet is re-suspended in a Tris 50 mM buffer medium at pH 7.4 and centrifuged at 500 g for 5 minutes at 4° C. The cells are lysed by sonication and centrifuged at 39,000 g for 10 minutes at 4° C. The pellet is re-suspended in the Tris 50 mM buffer medium at pH 7.4 and centrifuged at 50,000 g for 10 minutes at 4° C. The membranes obtained in this last pellet are stored at −80° C.

Measurement of the competitive inhibition of the binding of the [³H]-CP55940 to the CB2 receptors is carried out in duplicate using 96-well polypropylene plates. The cell membranes (10 μg of proteins/well) are incubated with the [³H]-CP55940 (1 nM) for 60 minutes at 25° C. in a Tris-HCl 50 mM buffer medium, pH 7.4, comprising 0.1% bovine serum albumin (BSA), 5 mM of MgCl₂, and 50 μg/ml of bacitracin.

The bound [³H]-CP55940 is separated from the free [³H]-CP55940 by filtration through GF/C glass fibre filter plates (Unifilter, Packard) pre-impregnated with 0.1% polyethylenimine (P.E.I.), using a Filtermate 196 (Packard). The filters are washed with Tris-HCl 50 mM buffer, pH 7.4 at 0-4° C. and the radioactivity present is determined using a counter (Packard Top Count).

The specific binding is obtained by subtracting the non-specific binding (determined in the presence of 0.1 μM of WIN55212-2 from the total binding). The data are analyzed by computer-assisted non-linear regression (MDL). For each test, a Cheng-Prusoff correction is made in order to convert the IC50 to the inhibition constant, Ki.

Thus,

$$Ki = \frac{IC 50}{1 + [ L ] / Kd}$$

where [L] is the concentration of the radioligand used in the test and Kd is the radioligand equilibrium dissociation constant.

Table 1 below shows the examples having an inhibition constant Ki of less than 5.10⁻⁶M and, among these examples, whose which have an inhibition constant of less than 50.10⁻⁹M.

The agonistic or antagonistic activity of the CB2 receptors of the compounds of the present invention was determined by measuring the production of cyclic AMP by the CHO-K1 cells transfected by the CB2 receptor.

### Measurement of the Production of Intracellular Cyclic AMP Via the CB2 Receptors:

The CHO-K1 cells expressing the CB2 cannabinoid receptors are cultured in 384-well plates in an RPMI 1640 medium with 10% foetal calf serum and 0.5 mg/ml of G418. The cells are washed twice with 50 μl of RPMI medium comprising 0.2% BSA and 0.5 mM of 3-isobutyl-1-methylxanthine (IBMX).

In order to measure the agonistic effect of a compound, the cells are incubated for 5 minutes at 37° C. in the presence of 0.5 mM of IBMX, then the stimulation of the production of cyclic AMP is obtained by adding 5 μM of Forskolin then the inhibition is measured by the addition of the compound at concentrations comprised between 1 pM and 10 μM in duplicate for 20 minutes at 37° C. The antagonistic effect of a compound is measured by inhibiting the inhibition of the production of cyclic AMP induced by WIN55212-2 in the presence of 5 μM of Forskolin, at concentrations comprised between 1 pM and 10 μM, in the presence of the compound to be tested, at concentrations comprised between 1 nM and 10 μM, in duplicate for 20 minutes at 37° C.

The reaction medium is eliminated and 80 μl of lysis buffer is added. The level of intracellular cyclic AMP is measured by a competition test with fluorescent cyclic AMP (CatchPoint, Molecular Devices).

TABLE 1

| Examples | Ki < 50 nM |
|---|---|
| 1 | x |
| 2 | x |
| 3 | |
| 4 | x |
| 5 | x |
| 6 | |
| 7 | |
| 8 | x |
| 9 | |
| 10 | |
| 11 | |
| 12 | x |
| 13 | |
| 14 | |
| 15 | |
| 16 | |
| 17 | x |
| 18 | |
| 19 | |
| 20 | |
| 21 | |
| 22 | |
| 23 | |
| 24 | |
| 26 | |
| 29 | |
| 30 | |
| 31 | x |
| 32 | |
| 33 | |
| 34 | |
| 35 | x |
| 37 | x |
| 38 | x |
| 39 | |
| 40 | |
| 41 | |
| 42 | |
| 43 | x |
| 44 | x |
| 45 | x |
| 46 | x |
| 47 | |
| 48 | x |

1. Compounds of general formula (I) 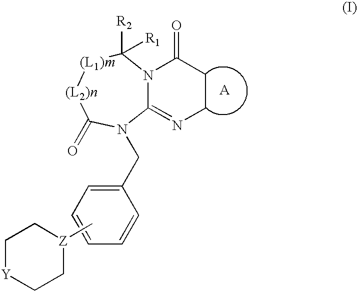
in racemic or enantiomeric form or any combinations of these forms and in which
L₁ represents 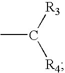
L₂ represents 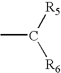
R₁, R₂, R₃, R₄, R₅ and R₆ represent, independently, a hydrogen atom or a hydroxy, halo, (C₁-C₈)alkyl, (C₁-C₈)haloalkyl, (C₂-C₈)alkenyl, (C₂-C₈)alkynyl, (C₃-Cₛ)cycloalkyl, (C₃-C₇)heterocycloalkyl, (C₁-C₆)alkoxy-(C₁-C₈)alkyl, (C₁-C₆)alkylthio-(C₁-C₈)alkyl, aryl, heteroaryl, aralkyloxy, aralkylthio radical or a —(CH₂)ₚ—R₇ radical, all these radicals being optionally substituted by one or more identical or different substituents chosen from: halo, nitro, hydroxy, oxo, amino, carboxamide, (C₁-C₆)alkylcarbonyl, (C₁-C₈)alkyl, (C₁-C₈)haloalkyl, (C₁-C₈)alkoxy, (C₁-C₈)haloalkoxy, aryl or aryloxy;
or R₁ and R₂, R₃ and R₄, or R₅ and R₆, together with the carbon atom to which they are attached, form a (C₃-C₈)cycloalkyl, (C₃-C₇)heterocycloalkyl, indanyl, tetrahydronaphthyl or decahydronaphthyl radical, these radicals being optionally substituted by one or more identical or different substituents chosen from: halo, hydroxy, (C₁-C₈)alkyl, (C₁-C₈)haloalkyl, (C₁-C₆)alkoxy;
or finally the R₃ and R₄ radicals, together with the adjacent R₁ and R₂ radicals on the one hand or R₅ and R₆ on the other hand, form a cycloalkenyl, heterocycloalkenyl, aryl or heteroaryl radical, these radicals being optionally substituted by one or more identical or different substituents chosen from halo, nitro, hydroxy, amino, carboxamide, (C₁-C₆)alkylcarbonyl, (C₁-C₈)alkyl, (C₁-C₈)haloalkyl, (C₁-C₈)alkoxy, (C₁-C₈)haloalkoxy;
p represents 1, 2, 3 or 4;
R₇ represents a —C(O)—O—(C₁-C₈)alkyl, —C(O)—NR_{N}R′_{N}, (C₃-C₈)cycloalkyl, (C₃-C₇)heterocycloalkyl, aryl or heteroaryl radical, all these radicals being optionally substituted by one or more identical or different substituents chosen from: halo, nitro, hydroxy, amino, (C₁-C₈)alkyl, (C₁-C₈)haloalkyl, (C₁-C₈)alkoxy, (C₁-C₈)haloalkoxy or aralkyloxy;
R_{N} and R′_{N} represent independently a hydrogen atom or a (C₁-C₈)alkyl, (C₁-C₈)haloalkyl, (C₂-C₈)alkenyl, (C₂-C₈)alkynyl, (C₃-C₈)cycloalkyl, (C₃-C₇)heterocycloalkyl, aryl or heteroaryl radical;
or R_{N} and R′_{N} together form a (C₃-C₇)heterocycloalkyl radical;
Z represents a nitrogen atom or a —CH— radical;
Y represents an oxygen atom, a —CHR₈ or —NR₈ radical;
R₈ represents a hydrogen atom, a (C₁-C₆)alkyl or (C₁-C₆)haloalkyl radical;
A represents an aromatic or non-aromatic, unsaturated, condensed, mono- or bi-cyclic ring containing optionally one or more identical or different heteroatoms chosen from O, S and N, and optionally substituted by one or more identical or different radicals, chosen from: halo, nitro, cyano, oxy, —X_{A}—Y_{A}, and aryl optionally substituted by one or more substituents chosen from: halo, (C₁-C₆)alkyl and (C₁-C₆)haloalkyl;
X_{A} represents a covalent bond, —O—, —S—, —C(O)—, —NR″_{N}—C(O)—, —C(O)—NR″_{N}—, —C(O)—O—, —SO₂— or —SO₂NH—;
Y_{A} represents the hydrogen atom or a (C₁-C₆)alkyl or (C₁-C₆)haloalkyl radical;
R″_{N} represents the hydrogen atom or a (C₁-C₆)alkyl radical;
n represents 0 or 1; m represents 0 or 1; or a pharmaceutically acceptable salt thereof.

2. Compounds according to claim 1, characterized in that A represents a ring chosen from: phenyl, naphthyl, thienyl, furyl, pyrrolyl, benzothienyl, benzofuryl, thieno-pyridinyl, indolyl and tetrahydrobenzo-thienyl; or a pharmaceutically acceptable salt thereof.

3. Compounds according to one of the previous claims, characterized in that A is optionally substituted by one or more identical or different radicals chosen from: halo, nitro, —X_{A}—Y_{A}, and phenyl;
X_{A} represents a covalent bond, —O— or —C(O);
Y_{A} represents the hydrogen atom or a (C₁-C₆)alkyl or (C₁-C₆)haloalkyl radical; or a pharmaceutically acceptable salt thereof.

4. Compounds according to one of the previous claims, characterized in that A represents a phenyl radical optionally substituted by one or more identical or different (C₁-C₆)alkyl or (C₁-C₆)alkoxy radicals; or a pharmaceutically acceptable salt thereof.

5. Compounds according to one of the previous claims, characterized in that Z represents a nitrogen atom; or a pharmaceutically acceptable salt thereof.

6. Compounds according to one of the previous claims, characterized in that Y represents the —NR₈ radical and R₈ represents a (C₁-C₆)alkyl radical; or a pharmaceutically acceptable salt thereof.

7. Compounds according to one of the previous claims, characterized in that they correspond to the following formula: 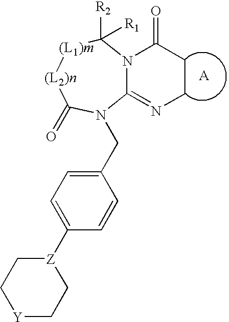
or a pharmaceutically acceptable salt thereof.

8. Compounds according to claim 1, characterized in that n represents 0; or a pharmaceutically acceptable salt thereof.

9. Compounds according to one of the previous claims, characterized in that m represents 0; or a pharmaceutically acceptable salt thereof.

10. Compounds according to one of the previous claims, characterized in that
n and m represent 0;
R₁ represents the hydrogen atom;
R₂ represents a hydrogen atom or a (C₁-C₈)alkyl, (C₃-C₈)cycloalkyl, (C₃-C₇)heterocycloalkyl or aryl radical optionally substituted by an aryl, heteroaryl radical, or a —(CH₂)ₚ—R₇ radical;
p represents 1 or 2;
R₇ represents a (C₃-C₈)cycloalkyl or aryl radical;
or R₁ and R₂, together with the carbon atom to which they are attached, form a (C₃-C₈)cycloalkyl or an indanyl radical; or a pharmaceutically acceptable salt thereof.

11. Compounds according to claim 10, characterized in that
m and n represent 0;
R₁ represents the hydrogen atom;
R₂ represents a (C₁-C₈)alkyl, (C₃-C₈)cycloalkyl, tetrahydropyranyl, naphthyl, phenyl radical optionally substituted by a phenyl, thienyl radical, or a —(CH₂)ₚ—R₇ radical;
p represents 1 or 2;
R₇ represents a cyclohexyl or phenyl radical;
or R₁ and R₂, together with the carbon atom to which they are attached, form a (C₃-C₈)cycloalkyl or an indanyl radical; or a pharmaceutically acceptable salt thereof.

12. Compounds according to one of claims 1 to 8, characterized in that m represents 1; or a pharmaceutically acceptable salt thereof.

13. Compounds according to one of claims 1 to 8, characterized in that
n represents 0 and m represents 1; and
either R₁, R₂, R₃ and R₄ represent, independently, a hydrogen atom;
or R₃ and R₄ represent, independently, a hydrogen atom; and
R₁ represents the hydrogen atom; R₂ represents a (C₁-C₈)alkyl, (C₃-C₈)cycloalkyl, tetrahydropyranyl, phenyl radical, or a —(CH₂)ₚ—R₇ radical; p represents 1 or 2 and R₇ represents a cyclohexyl or phenyl radical; or
R₁ and R₂ represent, independently, a (C₁-C₈)alkyl radical; or
R₁ and R₂, together with the carbon atom to which they are attached, form a (C₃-C₈)cycloalkyl.
or R₁ and R₂ represent independently a hydrogen atom;
R₃ represents the hydrogen atom; R₄ represents a (C₁-C₈)alkyl, (C₃-C₈)cycloalkyl, tetrahydropyranyl, phenyl radical, or a —(CH₂)ₚ—R₇ radical; p represents 1 or 2 and R₇ represents a cyclohexyl radical; or
R₃ and R₄ represent, independently, a (C₁-C₈)alkyl radical; or
R₃ and R₄, together with the carbon atom to which they are attached, form a (C₃-C₈)cycloalkyl;
or the radicals R₃ and R₄, together with the adjacent radicals R₁ and R₂, form a phenyl radical; or a pharmaceutically acceptable salt thereof.

14. Compounds according to one of claims 1 to 6, characterized in that n and m represent 1; or a pharmaceutically acceptable salt thereof.

15. Compounds according to one of claim 1 to 7, 12 or 14, characterized in that
m and n represent 1;
R₁, R₂, R₅ and R₆ represent, independently, a hydrogen atom or a (C₁-C₈)alkyl radical;
R₃ and R₄ represent, independently, a hydrogen atom or, together with the carbon atom to which they are attached, form a (C₃-Cₛ)cycloalkyl; or a pharmaceutically acceptable salt thereof.

16. Method for preparing a compound of formula (I) as defined in claim 1, characterized in that the chlorinated derivative (II) 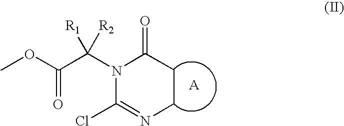
in which A, R₁ and R₂ are as defined in claim 1, is reacted with the amine (III) 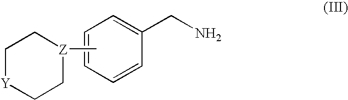
in which Y and Z are as defined in claim 1, in an aprotic solvent in the presence or absence of an inorganic base or tertiary amine, at a temperature of 18-80° C., in order to produce compound (I) in which n and m represent 0.

17. Method for preparing a compound of formula (I) as defined in claim 1, characterized in that the chlorinated derivative (IV) 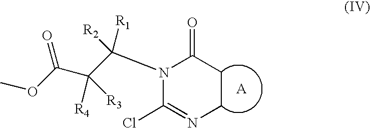
in which A, R₁, R₂, R₃ and R₄ are as defined in claim 1, is reacted with the amine (III) 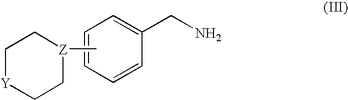
in which Y and Z are as defined in claim 1, in an aprotic solvent in the presence of an inorganic base or tertiary amine, at a temperature of 80 to 120° C., in order to produce compound (I) in which m and n represent 1 and 0 respectively.

18. Method for preparing a compound of formula (I) as defined in claim 1, characterized in that the chlorinated derivative (V) 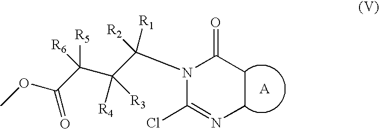
in which A, R₁, R₂, R₃, R₄, R₅ and R₆ are as defined in claim 1, is reacted with the amine (III) 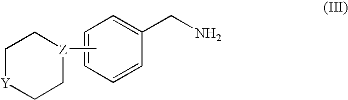
in which Y and Z are as defined in claim 1, in an aprotic solvent in the presence or absence of an organic base, in order to form the ester (VI) 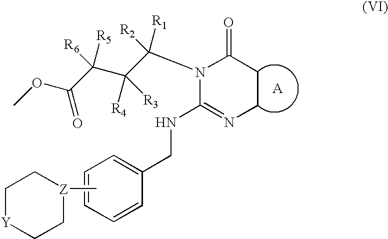
the ester (VI) thus formed is saponified in the presence of a base in an aprotic or protic solvent, at a temperature of 18 to 80° C., for 2 to 24 hours, in order to produce the corresponding acid (VII) 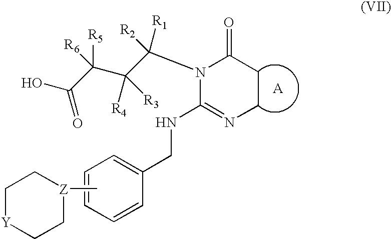
and finally the derivative (VII) is treated with a coupling agent, or with Mukaiyama's reagent in the presence of a tertiary amine, in an inert organic solvent, at ambient temperature for 3 to 24 hours, in order to produce compound (I) in which n and m represent 1.

19. Pharmaceutical compositions containing, as active ingredient, at least one product of formula I as defined in one of claims 1 to 15, or an addition salt with pharmaceutically to acceptable mineral or organic acids of said product of formula I, in combination with a pharmaceutically acceptable support.

20. Use of a compound according to one of claims 1 to 15, for preparing a medicament for the treatment of cell proliferation disorders, and preferably of cancer.

21. Use of a compound according to one of claims 1 to 15, for preparing a medicament for the treatment of immune disorders, inflammation, pain, osteoporosis, fibrosis, gastro-intestinal disorders, neurodegenerative diseases including multiple sclerosis and dyskinesia and Parkinson's disease.
